(19) United States
(12) Patent Application Publication
(10) Pub. No.: US 2021/0292255 A1
(43) Pub. Date: Sep. 23, 2021

(54) Yeast-Based Compositions for Enhancing Rhizosphere Properties and Plant Health

(71) Applicant: Locus Agriculture IP Company, LLC, Solon, OH (US)

(72) Inventors: Sean FARMER, Ft. Lauderdale, FL (US); Ken ALIBEK, Solon, OH (US); Paul ZORNER, Encinitas, CA (US); Samal IBRAGIMOVA, Solon, OH (US)

(21) Appl. No.: 17/264,831

(22) PCT Filed: Nov. 26, 2019
(86) PCT No.: PCT/US2019/063355

§ 371 Date: Jan. 31, 2021

Related U.S. Application Data
(60) Provisional application No. 62/771,703, filed on Nov. 27, 2018.

(51) Int. Cl. C05F 11/08; C05F 1/00; C12N 1/16; C12N 1/20

(52) U.S. Cl. CPC C05F 11/08; C12N 1/20; C12N 1/16; C05F 1/005

(57) ABSTRACT

Compositions and methods are provided for enhancing plant immunity, health, growth and yields, as well as enhancing rhizosphere properties, using beneficial microbes and/or their growth by-products. Specifically, the subject invention enhances plant health, growth and/or yields by applying a yeast-based composition to the plant (e.g., the roots) and/or its surrounding environment (e.g., the soil). Specifically, in one embodiment, the subject invention utilizes the killer yeast Wickerhamomyces anomalus and/or a species related closely thereto.

### CROSS-REFERENCE TO RELATED APPLICATION

This applications claims priority to U.S. Provisional Patent Application No. 62/771,703, filed Nov. 27, 2018, which is incorporated by reference herein in its entirety.

### BACKGROUND OF THE INVENTION

In the agriculture industry, certain common issues continue to hinder the ability of growers to maximize production yields while keeping costs low. These include, but are not limited to, infections and infestations caused by bacteria, fungi, nematodes and other pests and pathogens; the high costs of chemical fertilizers and herbicides, including their environmental and health impacts; and the difficulty for plants to efficiently absorb nutrients and water from different types of soil.

In citrus production, for example, widespread infection of citrus plants by pathogens such as those that cause citrus greening disease and citrus canker disease has led to significant hardships for citrus growers. Entire crops have been lost to these bacterial infections, leading to a decline in the production, and increase in price, of citrus products worldwide.

Citrus greening disease, which is also known is Huanglongbing (HLB) or yellow dragon disease, is an incurable infection caused by the Gram-negative bacterium Candidatus Liberibacter asiaticus. This disease has caused devastation for millions of acres of citrus crops throughout the United States and other parts of the world. Infected trees produce fruits that are green, misshapen and bitter, which are unsuitable for sale. The disease is spread by a disease-infected insect, the Asian citrus psyllid, and has put the future of the world's citrus trees at risk.

HLB lives in, and interferes with the function of, the phloem, or the plant vascular system that transports sugars to all parts of a tree. Thus, Liberibacter can move to and grow throughout an entire tree, including the roots. Before any expression of foliar symptoms, the infection typically has already caused significant damage to the root system, causing up to 50% loss in fibrous root density.

Root density continues to gradually decrease as symptoms develop in the canopy. This is probably due to plugging in the phloem, which restricts movement of sugars to the root system. Roots are crucial to a plant's ability to survive and grow. Loss of such a large percentage of the roots greatly reduces the immune health of the tree as well as its ability to absorb nutrients efficiently and to withstand water stress during extended dry periods. Thus, one of the most crucial characteristics for healthy crops is a healthy rhizosphere.

The rhizosphere is the zone of soil wherein a plant's root system grows and absorbs water and nutrients. To supplement soils with certain nutrients, many growers have relied heavily on the use of synthetic chemicals and chemical fertilizers for boosting crop yields and protecting crops from drought and disease. However, with reduced uptake capacity when, for example, a plant's root system is compromised, adding more water and/or nutrients to the soil may not lead to increased absorption by the root system. Instead, what is applied will flow through the rhizosphere and into the groundwater. As sources of pollution, responsible use of these substances is an ecological and commercial imperative. Over-dependence and long-term use of certain chemical fertilizers, pesticides and antibiotics can alter soil ecosystems, reduce stress tolerance, increase the prevalence of resistant pests, and impede plant growth and vitality.

Efficient nutrient and water absorption in the rhizosphere depends not only on the amount of water and nutrients present therein, but also upon the particular microbiome that exists within the soil. Soils contain billions of different microorganisms, which coexist with each other and with plants to form a complex network of relationships.

The optimum combination of microorganisms in a rhizosphere varies according to the type of plant as well as the type of soil in which it grows. No two plant species or regions will have the same network of microbes within a rhizosphere. Thus, while biological agents have the potential to play an increasingly vital role in crop health and soil remediation, treating a broad range of plant species over many different regions poses difficulties due to the complexity and specificity of each plant's optimal rhizospheric microbiome.

The economic costs and the adverse health and environmental impacts of current methods of crop production continue to burden the sustainability of crop-based consumer products. Thus, there is a continuing need for improved, non-toxic and environmentally-friendly methods of enhancing crop production at a low cost. In particular, there is a need for products to supplement soils for enhanced crop growth and yields, particularly in circumstances of compromised plant immune health.

### BRIEF SUMMARY OF THE INVENTION

The subject invention provides microbe-based products, as well as methods of using these microbe-based products in agricultural applications. Advantageously, the microbe-based products and methods of the subject invention are environmentally-friendly, non-toxic and cost-effective.

In preferred embodiments, the subject invention provides microbe-based soil treatment compositions and methods of their use for enhancing the health, growth and overall yields of crop plants by, for example, enhancing the health and/or growth of the plant's root system, as well as stimulating the plant's natural immune and other metabolic systems that contribute to plant health and productivity. In certain embodiments, the methods can improve the nutrient and/or moisture retention properties of the rhizosphere.

Advantageously, the soil treatment compositions of the subject invention can improve, for example, crop health, as well as crop growth and yields, even in situations where one or more of the plants in a crop are infected with a pathogen or where the immune health of the crop plants is otherwise compromised.

For example, in one embodiment, the subject invention can be used to improve health, growth and yields of citrus plants infected with, e.g., Candidatus Liberibacter asiaticus (citrus greening disease) and/or Xanthomonas axonopodis (citrus canker disease). Thus, in one embodiment, the subject invention can be used to improve the immune health and/or immune response of a plant.

In one embodiment, the subject invention provides soil treatment compositions comprising a microorganism and/or a growth by-product thereof. Also provided are methods of cultivating the microorganism and/or growth by-product.

In one embodiment, the soil treatment composition comprises a microorganisms characterized as a non-pathogenic yeast strain. Preferably, the composition comprises a non-pathogenic “killer yeast” strain, such as Wickerhamomyces anomalus, or other yeasts related thereto.

In one embodiment, the composition comprises one or more growth by-products or metabolites of the yeast. For example, W. anomalus is capable of producing a variety of metabolites, including enzymes such as phytase and exo beta-1, 3 glucanase, as well biosurfactants, including phospholipids and/or glycolipids.

In one embodiment, additional microorganisms can be included in the composition, provided they are compatible with the yeast and/or its growth by-products. The species and ratio of microorganisms and other ingredients in the composition can be determined according to, for example, the plant being treated, the soil type where the plant is growing, the health of the plant at the time of treatment, as well as other factors. Thus, the composition can be customizable for any given crop.

The microorganisms of the subject soil treatment compositions can be obtained through cultivation processes ranging from small to large scale. These cultivation processes include, but are not limited to, submerged cultivation/fermentation, solid state fermentation (SSF), and modifications, hybrids and/or combinations thereof. In preferred embodiments, the microbes are cultivated using SSF or modifications thereof.

The soil treatment composition can comprise the substrate leftover from fermentation and/or purified or unpurified growth by-products, such as biosurfactants, enzymes and/or other metabolites. The microbes can be live or inactive, although in preferred embodiments, the microbes are live.

The composition is preferably formulated for application to soil, seeds, whole plants, or plant parts (including, but not limited to, roots, tubers, stems, flowers and leaves). In certain embodiments, the composition is formulated as, for example, liquid, dust, granules, microgranules, pellets, wettable powder, flowable powder, emulsions, microcapsules, oils, or aerosols.

To improve or stabilize the effects of the composition, it can be blended with suitable adjuvants and then used as such or after dilution, if necessary. In certain embodiments, the composition is formulated as a concentrated liquid preparation, or as dry powder or dry granules that can be mixed with water and other components to form a liquid product. In one embodiment, the composition comprises the substrate, microbes and growth by-products, blended together and dried to form powder or granules.

In one embodiment, the composition can comprise glucose (e.g., in the form of molasses), glycerol, glycerin, and/or other osmoticum substances, to promote osmotic pressure during storage and transport of the dry product.

In one embodiment, methods are provided for enhancing plant health, growth and/or yields wherein a soil treatment composition comprising a yeast and/or growth by-products thereof is contacted with the plant and/or its surrounding environment. Preferably, the yeast is Wickerhamomyces anomalus or a species within the same genus and/or family.

In certain embodiments, the soil treatment composition is contacted with a plant part. In a specific embodiment, the composition is contacted with one or more roots of the plant. The composition can be applied directly to the roots, e.g., by spraying or dunking the roots, and/or indirectly, e.g., by administering the composition to the soil in which the plant grows (e.g., the rhizosphere). The composition can be applied to the seeds of the plant prior to or at the time of planting, or to any other part of the plant and/or its surrounding environment.

In one embodiment, the method can enhance plant health, growth and/or yields by enhancing the health and/or growth of the plant's roots. This can be achieved by, for example, improving the overall hospitability of the rhizosphere in which a plant's roots are growing. More specifically, in one embodiment, the methods can be used to improve the nutrient and/or moisture retention properties of the rhizosphere. In certain embodiments, the compositions and methods of the current invention facilitate nutrient uptake and/or water absorption by plants.

Additionally, in one embodiment, the method can be used to inoculate a plant's rhizosphere with a beneficial microorganism. For example, the yeast of the soil treatment composition can colonize the plant's rhizosphere and provide multiple benefits to the plant through the root-soil interface, including protection, nourishment, and, metabolic signaling that supports direct interaction between microbial and plant genomes.

In another embodiment, the method can be used to encourage beneficial microorganisms to colonize a rhizosphere. In yet another embodiment, the method can be used to fight off and/or discourage colonization of the rhizosphere by soil microorganisms that are deleterious or that might compete with beneficial soil microorganisms.

Furthermore, in one embodiment, the method can be used to provide a nutrient to a plant, and/or to treat and/or prevent a nutrient deficiency in a plant. For example, in one embodiment, when the yeast of the soil treatment composition are inactive, or when they die, their cells provide an abundance of nutrients, proteins, vitamins, and minerals for the plants and/or for other soil microbiota to utilize.

In another embodiment, the method can be used to provide the plant with phosphorus in the form of phosphates. This is because W. anomalus can produce phytase, an enzyme that is capable of converting phytic acid present in soil into plant-bioavailable (e.g., root-absorbable) phosphates. Accordingly, the method can be used to treat and/or prevent a phosphorus deficiency in a plant.

Advantageously, the subject methods can be used to enhance health, growth and/or yields in plants having compromised immune health due to an infection from a pathogenic or a biotic agent, or from an environmental stressor, such as, for example, drought. Thus, the subject methods can also be used for improving the immune health, or immune response, of plants.

In certain embodiments, the microorganisms of the composition work synergistically with the other ingredients and/or optional additional microorganisms to enhance health, growth and/or yields of plants.

The compositions and methods of the subject invention can be used either alone or in combination with other compounds and/or methods for efficiently enhancing plant health, growth and/or yields, and/or for supplementing the growth of the first and second microbes. For example, in one embodiment, the composition can include and/or can be applied concurrently with nutrients and/or micronutrients for enhancing plant and/or microbe growth, such as magnesium, phosphate, nitrogen, potassium, selenium, calcium, sulfur, iron, copper, and zinc; one or more nano-fertilizers, such Aqua-Yield, NanoGro™; and/or prebiotics, such as kelp extract, fulvic acid, chitin, humate and/or humic acid. The exact materials and the quantities thereof can be determined by a grower or an agricultural scientist having the benefit of the subject disclosure.

The compositions and methods can also be used in combination with other crop management systems. In one embodiment, the composition can optionally comprise, or be applied with, natural and/or chemical pesticides and/or repellants, such as, for example, any known commercial and/or homemade pesticide that is compatible with the combination of microorganisms being applied. In some embodiments, the composition can also comprise, or be applied with, for example, herbicides, fertilizers, and/or other compatible soil amendments, including commercial products containing nutrient sources (e.g., nitrogen-phosphorous-potassium (NPK) and/or micronutrients).

Advantageously, the present invention can be used without releasing large quantities of inorganic compounds into the environment. Additionally, the compositions and methods utilize components that are biodegradable and toxicologically safe. Thus, the present invention can be used as a “green” soil treatment.

### BRIEF DESCRIPTION OF THE FIGURES

FIG. 1 shows the results of a comparison study between young citrus trees treated with a composition comprising W. anomalus (“STR10”) and untreated control trees. Height, trunk caliper and growth index (GI) were measured over a 6 month period after treatment.

FIG. 2 shows the results of a comparison study between young citrus trees treated with a composition comprising W. anomalus (“STR10”) and untreated control trees, wherein tree height was measured over an 18-month period.

FIG. 3 shows the results of a comparison study between young citrus trees treated with a composition comprising W. anomalus (“STR10”) and untreated control trees, wherein Growth Index (GI) was measured over an 18-month period.

FIG. 4 shows the results of a comparison study between young citrus trees treated with a composition comprising W. anomalus (“STR10”) and untreated control trees, wherein trunk caliper was measured over an 18-month period.

FIGS. 5A-5B show the results of a comparison study between young citrus trees treated with a composition comprising W. anomalus (“STR10”) and untreated control trees, wherein the percentage of trees exhibiting new shoot growth (5A) and the average shoot count per tree (5B) were measured.

FIG. 6 shows the results of a comparison study between young citrus trees treated with a composition comprising W. anomalus (“STR10”) and untreated control trees, wherein average fruit count per tree was measured.

FIGS. 7A-7B show the results of comparison studies between lettuce plants treated with a composition comprising W. anomalus (“STR10”) and untreated control plants (7A), and between lettuce plants treated with STR10 plus a composition comprising Trichoderma harzianum and Bacillus amyloliquefaciens (“ThBa”) and untreated control plants (7B), wherein the average weight (g) of lettuce heads were measured.

FIGS. 8A-8B show the results of a comparison study between peanut plants treated with a composition comprising W. anomalus (“STR10”) and untreated control plants, wherein the average flower count per 30 sq. ft. (8A) and the average canopy size (in.) (8B) were measured.

FIGS. 9A-9B show the results of comparison studies between zucchini plants treated with a composition comprising W. anomalus (“STR10”), STR10 plus a composition comprising Trichoderma harzianum and Bacillus amyloliquefaciens (“ThBa”), and untreated control plants, wherein the nitrogen content (9A) and the magnesium content (9B) of leaf tissue were measured.

### DETAILED DESCRIPTION OF THE INVENTION

The subject invention provides microbe-based products, as well as methods of using these microbe-based products in agricultural applications. Advantageously, the microbe-based products and methods of the subject invention are environmentally-friendly, non-toxic and cost-effective.

In preferred embodiments, the subject invention provides microbe-based soil treatment compositions and methods of their use for enhancing the health, growth and overall yields of crop plants by, for example, improving the nutrient and moisture retention properties of the rhizosphere. Advantageously, the soil treatment compositions of the subject invention can improve, for example, crop health, as well as crop growth and yields, even in situations where one or more of the plants in a crop are infected with a pathogen or where the immune health of the crop plants is otherwise compromised. For example, in one embodiment, the subject invention can be used to improve health, growth and/or yields of citrus plants infected with, e.g., Candidatus Liberibacter asiaticus (citrus greening disease) and/or Xanthomonas axonopodis (citrus canker disease).

### Selected Definitions

The subject invention utilizes “microbe-based compositions,” meaning a composition that comprises components that were produced as the result of the growth of microorganisms or other cell cultures. Thus, the microbe-based composition may comprise the microbes themselves and/or by-products of microbial growth. The microbes may be in a vegetative state, in spore or conidia form, in hyphae form, in any other form of propagule, or a mixture of these. The microbes may be planktonic or in a biofilm form, or a mixture of both. The by-products of growth may be, for example, metabolites, cell membrane components, expressed proteins, and/or other cellular components. The microbes may be intact or lysed. In preferred embodiments, the microbes are present, with growth medium in which they were grown, in the microbe-based composition. The microbes may be present at, for example, a concentration of at least 1×10⁴, 1×10⁵, 1×10⁶, 1×10⁷, 1×10⁸, 1×10⁹, 1×10¹⁰, 1×10¹¹, 1×10¹² or 1×10¹³ or more CFU per gram or per ml of the composition.

The subject invention further provides “microbe-based products,” which are products that are to be applied in practice to achieve a desired result. The microbe-based product can be simply the microbe-based composition harvested from the microbe cultivation process. Alternatively, the microbe-based product may comprise further ingredients that have been added. These additional ingredients can include, for example, stabilizers, buffers, appropriate carriers, such as water, salt solutions, or any other appropriate carrier, added nutrients to support further microbial growth, non-nutrient growth enhancers and/or agents that facilitate tracking of the microbes and/or the composition in the environment to which it is applied. The microbe-based product may also comprise mixtures of microbe-based compositions. The microbe-based product may also comprise one or more components of a microbe-based composition that have been processed in some way such as, but not limited to, filtering, centrifugation, lysing, drying, purification and the like.

As used herein, “harvested” in the context of fermentation of a microbe-based composition refers to removing some or all of the microbe-based composition from a growth vessel.

As used herein, a “biofilm” is a complex aggregate of microorganisms, such as bacteria, wherein the cells adhere to each other. The cells in biofilms are physiologically distinct from planktonic cells of the same organism, which are single cells that can float or swim in liquid medium.

As used herein, an “isolated” or “purified” compound is substantially free of other compounds, such as cellular material, with which it is associated in nature. A purified or isolated polynucleotide (ribonucleic acid (RNA) or deoxyribonucleic acid (DNA)) is free of the genes or sequences that flank it in its naturally-occurring state. A purified or isolated polypeptide is free of the amino acids or sequences that flank it in its naturally-occurring state. “Isolated” in the context of a microbial strain means that the strain is removed from the environment in which it exists in nature. Thus, the isolated strain may exist as, for example, a biologically pure culture, or as spores (or other forms of the strain) in association with a carrier.

As used herein, a “biologically pure culture” is a culture that has been isolated from materials with which it is associated in nature. In a preferred embodiment, the culture has been isolated from all other living cells. In further preferred embodiments, the biologically pure culture has advantageous characteristics compared to a culture of the same microbe as it exists in nature. The advantageous characteristics can be, for example, enhanced production of one or more growth by-products.

In certain embodiments, purified compounds are at least 60% by weight (dry weight) the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis.

A “metabolite” refers to any substance produced by metabolism (e.g., a growth by-product) or a substance necessary for taking part in a particular metabolic process. A metabolite can be an organic compound that is a starting material (e.g., glucose), an intermediate (e.g., acetyl-CoA) in, or an end product (e.g., n-butanol) of metabolism. Examples of metabolites include, but are not limited to, biosurfactants, biopolymers, enzymes, acids, solvents, alcohols, proteins, vitamins, minerals, microelements, and amino acids.

As used herein, “modulate” means to cause an alteration (e.g., increase or decrease). Such alterations are detected by standard art known methods.

Ranges provided herein are understood to be shorthand for all of the values within the range. For example, a range of 1 to 20 is understood to include any number, combination of numbers, or sub-range from the group consisting of 1, 2, 3, 4, 5, 6, 7, 8, 9, 10, 11, 12, 13, 14, 15, 16, 17, 18, 19, 20, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

As used herein, “reduce” refers to a negative alteration, and the term “increase” refers to a positive alteration, each of at least 1%, 5%, 10%, 25%, 50%, 75%, or 100%.

As used herein, “reference” refers to a standard or control condition.

As used herein, “surfactant” refers to a compound that lowers the surface tension (or interfacial tension) between two liquids or between a liquid and a solid. Surfactants act as, e.g., detergents, wetting agents, emulsifiers, foaming agents, and dispersants. A “biosurfactant” is a surfactant produced by a living organism.

As used herein, “agriculture” means the cultivation and breeding of plants, algae and/or fungi for food, fiber, biofuel, medicines, cosmetics, supplements, ornamental purposes and other uses.

According to the subject invention, agriculture can also include horticulture, landscaping, gardening, plant conservation, forestry and reforestation, pasture and prairie restoration, orcharding, arboriculture, and agronomy. Further included in agriculture is the care, monitoring and maintenance of soil.

As used herein, “enhancing” means improving or increasing. For example, enhanced plant health means improving the plant's ability grow and thrive, which includes increased seed germination and/or emergence, improved ability to ward off pests and/or diseases, and improved ability to survive environmental stressors, such, as droughts and/or overwatering. Enhanced plant growth and/or enhanced plant biomass means increasing the size and/or mass of a plant both above and below the ground (e.g., increased canopy/foliar volume, height, trunk caliper, branch length, shoot length, protein content, root size/density and/or overall growth index), and/or improving the ability of the plant to reach a desired size and/or mass. Enhanced yields mean improving the end products produced by the plants in a crop, for example, by increasing the number and/or size of fruits, leaves, roots and/or tubers per plant, and/or improving the quality of the fruits, leaves, roots and/or tubers (e.g., improving taste, texture, brix, chlorophyll content and/or color).

As used herein “preventing” or “prevention” of a situation or occurrence means delaying, inhibiting, suppressing, forestalling, and/or minimizing the onset, extensiveness or progression of the situation or occurrence. Prevention can include, but does not require, indefinite, absolute or complete prevention, meaning the sign or symptom may still develop at a later time. Prevention can include reducing the severity of the onset of such a disease, condition or disorder, and/or inhibiting the progression of the condition or disorder to a more severe condition or disorder.

As used herein, the term “control” used in reference to a pest means killing, disabling, immobilizing, or reducing population numbers of a pest, or otherwise rendering the pest substantially incapable of causing harm.

As used herein, a “pest” is any organism, other than a human, that is destructive, deleterious and/or detrimental to humans or human concerns (e.g., agriculture, horticulture). In some, but not all instances, a pest may be a pathogenic organism. Pests may cause or be a vector for infections, infestations and/or disease, or they may simply feed on or cause other physical harm to living tissue. Pests may be single- or multi-cellular organisms, including but not limited to, viruses, fungi, bacteria, parasites, protozoa and/or nematodes.

As used herein, a “soil amendment” or a “soil conditioner” is any compound, material, or combination of compounds or materials that are added into soil to enhance the properties of the soil and/or rhizosphere. Soil amendments can include organic and inorganic matter, and can further include, for example, fertilizers, pesticides and/or herbicides. Nutrient-rich, well-draining soil is essential for the growth and health of plants, and thus, soil amendments can be used for enhancing the plant biomass by altering the nutrient and moisture content of soil. Soil amendments can also be used for improving many different qualities of soil, including but not limited to, soil structure (e.g., preventing compaction); improving the nutrient concentration and storage capabilities; improving water retention in dry soils; and improving drainage in waterlogged soils.

As used herein, an “abiotic stressor” is a non-living condition that has a negative impact on a living organism in a specific environment. The abiotic stressor must influence the environment beyond its normal range of variation to adversely affect the population performance or individual physiology of the organism in a significant way. Examples of abiotic stressors include, but are not limited to, drought, extreme temperatures (high or low), flood, high winds, natural disasters (e.g., hurricanes, avalanches, tornadoes), soil pH changes, high radiation, compaction of soil, pollution, and others. Alternatively, a “biotic stressor” is damaging and/or harmful action towards a living organism by another living organism. Biotic stressors can include, for example, damage and/or disease caused by a pest, competition with other organisms for resources and/or space, and various human activities.

The transitional term “comprising,” which is synonymous with “including,” or “containing,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. Use of the term “comprising” contemplates other embodiments that “consist” or “consist essentially” of the recited component(s).

Unless specifically stated or obvious from context, as used herein, the term “or” is understood to be inclusive. Unless specifically stated or obvious from context, as used herein, the terms “a,” “and” and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

All references cited herein are hereby incorporated by reference in their entirety.

### Soil Treatment Compositions

In one embodiment, the subject invention provides soil treatment compositions comprising a microorganism and/or a growth by-product thereof. The soil treatment composition can be used to enhance plant health, growth and/or yields, even in plants that have been infected by a pathogen or disease. More specifically, the subject compositions can be used to enhance plant root health and/or growth, and/or to enhance the immune health of a plant. In certain embodiments, the soil treatment composition can also be used to inoculate plant roots, and/or the rhizosphere in which the roots grow, with a beneficial microorganism.

Advantageously, in preferred embodiments, the microbe-based compositions according to the subject invention are non-toxic and can be applied in high concentrations without causing irritation to, for example, the skin or digestive tract of a human or other non-pest animal. Thus, the subject invention is particularly useful where application of the microbe-based compositions occurs in the presence of living organisms, such as growers and livestock.

In one embodiment, the soil treatment composition comprises a non-pathogenic yeast. Preferably, the composition comprises a non-pathogenic “killer yeast,” such as Wickerhamomyces anomalus, or other yeasts related thereto.

In one embodiment, the composition comprises one or more growth by-products or metabolites of the yeast. For example, W. anomalus is capable of producing a variety of metabolites, including enzymes such as phytase and exo beta-1, 3 glucanase, as well as biosurfactants, including phospholipids and/or glycolipids.

In some embodiments, the composition can further comprise one or more additional microbes that can be useful for enhancing rhizosphere properties and/or enhancing plant health. The species and ratio of additional microorganisms and/or other ingredients in the composition can be customized according to, for example, the plant being treated, the soil type where the plant is growing, the health of the plant at the time of treatment, as well as other factors. Thus, the composition can be tailored for any given crop.

For example, in one embodiment, the composition further comprises a Trichoderma spp. fungus, such as, for example, T. harzianum, T. viride, T. hamatum, and/or T. reesei). In one embodiment, the composition further comprises a Bacillus spp. bacterium, such as, for example, B. subtilis and/or B. amyloliquefaciens.

In one embodiment, the composition further comprises a microorganism capable of fixing, solubilizing and/or mobilizing nitrogen, potassium, phosphorous (or phosphate) and/or micronutrients in soil. In one embodiment, a nitrogen-fixing bacteria can be included, such as, for example, Azotobacter vinelandii. In another embodiment, a potassium-mobilizing bacteria can be included, such as, for example, Frateuria aurantia.

In one embodiment, the microorganism or combination of microorganisms of the subject composition comprise about 5 to 20% of the total composition by weight, or about 8 to 15%, or about 10 to 12%. In one embodiment, the composition comprises about 1×10⁶ to 1×10¹², 1×10⁷ to 1×10¹¹, 1×10⁸ to 1×10¹⁰, or 1×10⁹ CFU/ml of each microorganism.

The combination and ratio of species of microorganisms and other ingredients in the composition can be customized in accordance with, for example, the plant being treated, the soil type where the plant is growing, the health of the plant at the time of treatment, as well as other factors.

The microbes and microbe-based compositions of the subject invention have a number of beneficial properties that are useful for enhancing plant health, growth and/or yields. For example, the compositions can comprise products resulting from the growth of the microorganisms, such as biosurfactants, proteins and/or enzymes, either in purified or crude form.

In one embodiment, the microorganisms of the subject composition are capable of producing a biosurfactant. In another embodiment, biosurfactants can be produced separately by other microorganisms and added to the composition, either in purified form or in crude form. Crude form biosurfactants can comprise, for example, biosurfactants and other products of cellular growth in fermentation medium resulting from cultivation of a biosurfactant-producing microbe. This crude form biosurfactant composition can comprise from about 0.001% to about 90%, about 25% to about 75%, about 30% to about 70%, about 35% to about 65%, about 40% to about 60%, about 45% to about 55%, or about 50% pure biosurfactant.

Biosurfactants form an important class of secondary metabolites produced by a variety of microorganisms such as bacteria, fungi, and yeasts. As amphiphilic molecules, microbial biosurfactants reduce the surface and interfacial tensions between the molecules of liquids, solids, and gases. Furthermore, the biosurfactants according to the subject invention are biodegradable, have low toxicity, are effective in solubilizing and degrading insoluble compounds in soil and can be produced using low cost and renewable resources. They can inhibit adhesion of undesirable microorganisms to a variety of surfaces, prevent the formation of biofilms, and can have powerful emulsifying and demulsifying properties. Furthermore, the biosurfactants can also be used to improve wettability and to achieve even solubilization and/or distribution of fertilizers, nutrients, and water in the soil.

Biosurfactants according to the subject methods can be selected from, for example, low molecular weight glycolipids (e.g., sophorolipids, cellobiose lipids, rhamnolipids, mannosylerythritol lipids and trehalose lipids), lipopeptides (e.g., surfactin, iturin, fengycin, arthrofactin and lichenysin), flavolipids, phospholipids (e.g., cardiolipins), and high molecular weight polymers such as lipoproteins, lipopolysaccharide-protein complexes, and polysaccharide-protein-fatty acid complexes.

The composition can comprise one or more biosurfactants at a concentration of 0.001% to 10%, 0.01% to 5%, 0.05% to 2%, and/or from 0.1% to 1%.

Advantageously, in accordance with the subject invention, the soil treatment composition may comprise the medium in which each of the microorganism were grown. The composition may be, for example, at least, by weight, 1%, 5%, 10%, 25%, 50%, 75%, or 100% growth medium.

The fermentation medium can contain a live and/or an inactive culture, purified or crude form growth by-products, such as biosurfactants, enzymes, and/or other metabolites, and/or any residual nutrients. The amount of biomass in the composition, by weight, may be, for example, anywhere from about 0.01% to 100%, about 1% to 90%, about 5% to about 80%, or about 10% to about 75%.

The product of fermentation may be used directly, with or without extraction or purification. If desired, extraction and purification can be easily achieved using standard extraction and/or purification methods or techniques described in the literature.

In one embodiment, when a combination of strains of microorganism are included in the composition, the different strains of microbe are grown separately and then mixed together to produce the soil treatment composition.

In one embodiment, the composition is preferably formulated for application to soil, seeds, whole plants, or plant parts (including, but not limited to, roots, tubers, stems, flowers and leaves). In certain embodiments, the composition is formulated as, for example, liquid, dust, granules, microgranules, pellets, wettable powder, flowable powder, emulsions, microcapsules, oils, or aerosols.

To improve or stabilize the effects of the composition, it can be blended with suitable adjuvants and then used as such or after dilution, if necessary. In preferred embodiments, the composition is formulated as a liquid, a concentrated liquid, or as dry powder or granules that can be mixed with water and other components to form a liquid product.

In one embodiment, the composition can comprise glucose (e.g., in the form of molasses), glycerol and/or glycerin, as, or in addition to, an osmoticum substance, to promote osmotic pressure during storage and transport of the dry product.

The compositions can be used either alone or in combination with other compounds and/or methods for efficiently enhancing plant health, growth and/or yields, and/or for supplementing the growth of the first and second microbes. For example, in one embodiment, the composition can include and/or can be applied concurrently with nutrients and/or micronutrients for enhancing plant and/or microbe growth, such as magnesium, phosphate, nitrogen. potassium, selenium, calcium, sulfur, iron, copper, and zinc: and/or one or more prebiotics, such as kelp extract, fulvic acid, chitin, humate and/or humic acid. The exact materials and the quantities thereof can be determined by a grower or an agricultural scientist having the benefit of the subject disclosure.

The compositions can also be used in combination with other agricultural compounds and/or crop management systems. In one embodiment, the composition can optionally comprise, or be applied with, for example, natural and/or chemical pesticides (e.g., azoxystrobin, ipconazole, metalaxyl, trifloxystrobin, clothiandin, VOTiVO, thiamethoxam, cyantaniliprole, fludioxonil, tioxazafen, glycolipids, lipopeptides, deet, diatomaceous earth, citronella, essential oils, mineral oils, garlic extract, chili extract), repellants, herbicides, fertilizers, water treatments, non-ionic surfactants and/or soil amendments that are compatible with the microorganism or combination of microorganisms being applied.

Further components can be added to the composition, for example, buffering agents, carriers, other microbe-based compositions produced at the same or different facility, viscosity modifiers, preservatives, nutrients for microbe growth, tracking agents, biocide, other microbes, surfactants, emulsifying agents, lubricants, solubility controlling agents, pH adjusting agents, preservatives, stabilizers and ultra-violet light resistant agents.

The pH of the microbe-based composition should be suitable for the microorganism of interest. In a preferred embodiment, the pH of the final microbe-based composition ranges from 3.0 to 8.0, or about 3.5 to 7.0.

Optionally, the composition can be stored prior to use. The storage time is preferably short. Thus, the storage time may be less than 60 days, 45 days, 30 days, 20 days, 15 days, 10 days, 7 days, 5 days, 3 days, 2 days, 1 day, or 12 hours. In a preferred embodiment, if live cells are present in the product, the product is stored at a cool temperature such as, for example, less than 20° C., 15° C., 10° C., or 5° C.

The microbe-based compositions may be used without further stabilization, preservation, and storage, however. Advantageously, direct usage of these microbe-based compositions preserves a high viability of the microorganisms, reduces the possibility of contamination from foreign agents and undesirable microorganisms, and maintains the activity of the by-products of microbial growth.

The microbe-based compositions may be used without further stabilization, preservation, and storage, however. Advantageously, direct usage of these microbe-based compositions preserves a high viability of the microorganisms, reduces the possibility of contamination from foreign agents and undesirable microorganisms, and maintains the activity of the by-products of microbial growth.

In other embodiments, the composition (microbes, growth medium, or microbes and medium) can be placed in containers of appropriate size, taking into consideration, for example, the intended use, the contemplated method of application, the size of the fermentation vessel, and any mode of transportation from microbe growth facility to the location of use. Thus, the containers into which the microbe-based composition is placed may be, for example, from 1 pint to 1,000 gallons or more. In certain embodiments the containers are 1 gallon, 2 gallons, 5 gallons, 25 gallons, or larger.

### Growth of Microbes According to the Subject Invention

The subject invention utilizes methods for cultivation of microorganisms and production of microbial metabolites and/or other by-products of microbial growth. The subject invention further utilizes cultivation processes that are suitable for cultivation of microorganisms and production of microbial metabolites on a desired scale. These cultivation processes include, but are not limited to, submerged cultivation/fermentation, solid state fermentation (SSF), and modifications, hybrids and/or combinations thereof.

As used herein “fermentation” refers to cultivation or growth of cells under controlled conditions. The growth could be aerobic or anaerobic. In preferred embodiments, the microorganisms are grown using SSF and/or modified versions thereof.

In one embodiment, the subject invention provides materials and methods for the production of biomass (e.g., viable cellular material), extracellular metabolites (e.g. small molecules and excreted proteins), residual nutrients and/or intracellular components (e.g. enzymes and other proteins).

The microbe growth vessel used according to the subject invention can be any fermenter or cultivation reactor for industrial use. In one embodiment, the vessel may have functional controls/sensors or may be connected to functional controls/sensors to measure important factors in the cultivation process, such as pH, oxygen, pressure, temperature, humidity, microbial density and/or metabolite concentration.

In a further embodiment, the vessel may also be able to monitor the growth of microorganisms inside the vessel (e.g., measurement of cell number and growth phases). Alternatively, a daily sample may be taken from the vessel and subjected to enumeration by techniques known in the art, such as dilution plating technique. Dilution plating is a simple technique used to estimate the number of organisms in a sample. The technique can also provide an index by which different environments or treatments can be compared.

In one embodiment, the method includes supplementing the cultivation with a nitrogen source. The nitrogen source can be, for example, potassium nitrate, ammonium nitrate ammonium sulfate, ammonium phosphate, ammonia, urea, and/or ammonium chloride. These nitrogen sources may be used independently or in a combination of two or more.

The method can provide oxygenation to the growing culture. One embodiment utilizes slow motion of air to remove low-oxygen containing air and introduce oxygenated air. In the case of submerged fermentation, the oxygenated air may be ambient air supplemented daily through mechanisms including impellers for mechanical agitation of liquid, and air spargers for supplying bubbles of gas to liquid for dissolution of oxygen into the liquid.

The method can further comprise supplementing the cultivation with a carbon source. The carbon source is typically a carbohydrate, such as glucose, sucrose, lactose, fructose, trehalose, mannose, mannitol, and/or maltose; organic acids such as acetic acid, fumaric acid, citric acid, propionic acid, malic acid, malonic acid, and/or pyruvic acid; alcohols such as ethanol, propanol, butanol, pentanol, hexanol, isobutanol, and/or glycerol; fats and oils such as soybean oil, canola oil, rice bran oil, olive oil, corn oil, sesame oil, and/or linseed oil; etc. These carbon sources may be used independently or in a combination of two or more.

In one embodiment, growth factors and trace nutrients for microorganisms are included in the medium. This is particularly preferred when growing microbes that are incapable of producing all of the vitamins they require. Inorganic nutrients, including trace elements such as iron, zinc, copper, manganese, molybdenum and/or cobalt may also be included in the medium. Furthermore, sources of vitamins, essential amino acids, and microelements can be included, for example, in the form of flours or meals, such as corn flour, or in the form of extracts, such as yeast extract, potato extract, beef extract, soybean extract, banana peel extract, and the like, or in purified forms. Amino acids such as, for example, those useful for biosynthesis of proteins, can also be included.

In one embodiment, inorganic salts may also be included. Usable inorganic salts can be potassium dihydrogen phosphate, dipotassium hydrogen phosphate, disodium hydrogen phosphate, magnesium sulfate, magnesium chloride, iron sulfate, iron chloride, manganese sulfate, manganese chloride, zinc sulfate, lead chloride, copper sulfate, calcium chloride, sodium chloride, calcium carbonate, and/or sodium carbonate. These inorganic salts may be used independently or in a combination of two or more.

In some embodiments, the method for cultivation may further comprise adding additional acids and/or antimicrobials in the medium before, and/or during the cultivation process. Antimicrobial agents or antibiotics are used for protecting the culture against contamination.

Additionally, antifoaming agents may also be added to prevent the formation and/or accumulation of foam during submerged cultivation.

The pH of the mixture should be suitable for the microorganism of interest. Buffers, and pH regulators, such as carbonates and phosphates, may be used to stabilize pH near a preferred value. When metal ions are present in high concentrations, use of a chelating agent in the medium may be necessary.

The microbes can be grown in planktonic form or as biofilm. In the case of biofilm, the vessel may have within it a substrate upon which the microbes can be grown in a biofilm state. The system may also have, for example, the capacity to apply stimuli (such as shear stress) that encourages and/or improves the biofilm growth characteristics.

In one embodiment, the method for cultivation of microorganisms is carried out at about 5° to about 100° C., preferably, 15 to 60° C., more preferably, 25 to 50° C. In a further embodiment, the cultivation may be carried out continuously at a constant temperature. In another embodiment, the cultivation may be subject to changing temperatures.

In one embodiment, the equipment used in the method and cultivation process is sterile. The cultivation equipment such as the reactor/vessel may be separated from, but connected to, a sterilizing unit, e.g., an autoclave. The cultivation equipment may also have a sterilizing unit that sterilizes in situ before starting the inoculation. Air can be sterilized by methods know in the art. For example, the ambient air can pass through at least one filter before being introduced into the vessel. In other embodiments, the medium may be pasteurized or, optionally, no heat at all added, where the use of low water activity and low pH may be exploited to control undesirable bacterial growth.

In one embodiment, the subject invention further provides a method for producing microbial metabolites such as, for example, biosurfactants, enzymes, proteins, ethanol, lactic acid, beta-glucan, peptides, metabolic intermediates, polyunsaturated fatty acid, and lipids, by cultivating a microbe strain of the subject invention under conditions appropriate for growth and metabolite production; and, optionally, purifying the metabolite. The metabolite content produced by the method can be, for example, at least 20%, 30%, 40%, 50%, 60%, 70%, 80%, or 90%.

The microbial growth by-product produced by microorganisms of interest may be retained in the microorganisms or secreted into the growth medium. The medium may contain compounds that stabilize the activity of microbial growth by-product.

The biomass content of the fermentation medium may be, for example, from 5 g/l to 180 g/l or more, or from 10 g/I to 150 g/l.

The cell concentration may be, for example, at least 1×10⁶ to 1×10¹², 1×10⁷ to 1×10¹¹, 1×10⁸ to 1×10¹⁰, or 1×10⁹ CFU/ml.

The method and equipment for cultivation of microorganisms and production of the microbial by-products can be performed in a batch, a quasi-continuous process, or a continuous process.

In one embodiment, all of the microbial cultivation composition is removed upon the completion of the cultivation (e.g., upon, for example, achieving a desired cell density, or density of a specified metabolite). In this batch procedure, an entirely new batch is initiated upon harvesting of the first batch.

In another embodiment, only a portion of the fermentation product is removed at any one time. In this embodiment, biomass with viable cells, spores, conidia, hyphae and/or mycelia remains in the vessel as an inoculant for a new cultivation batch. The composition that is removed can be a cell-free medium or contain cells, spores, or other reproductive propagules, and/or a combination of thereof. In this manner, a quasi-continuous system is created.

Advantageously, the method does not require complicated equipment or high energy consumption. The microorganisms of interest can be cultivated at small or large scale on site and utilized, even being still-mixed with their media.

Advantageously, the microbe-based products can be produced in remote locations. The microbe growth facilities may operate off the grid by utilizing, for example, solar, wind and/or hydroelectric power.

### Microbial Strains

The microorganisms useful according to the subject invention can be, for example, non-plant-pathogenic strains of bacteria, yeast and/or fungi. These microorganisms may be natural, or genetically modified microorganisms. For example, the microorganisms may be transformed with specific genes to exhibit specific characteristics. The microorganisms may also be mutants of a desired strain. As used herein, “mutant” means a strain, genetic variant or subtype of a reference microorganism, wherein the mutant has one or more genetic variations (e.g., a point mutation, missense mutation, nonsense mutation, deletion, duplication, frameshift mutation or repeat expansion) as compared to the reference microorganism. Procedures for making mutants are well known in the microbiological art. For example, UV mutagenesis and nitrosoguanidine are used extensively toward this end.

In one embodiment, the microorganism is a yeast or fungus. Yeast and fungus species suitable for use according to the current invention, include Aureobasidium (e.g., A. pullulans), Blakeslea, Candida (e.g., C. apicola, C. bombicola, C. nodaensis), Cryptococcus, Debaryomyces (e.g., D. hansenii), Entomophthora, Hanseniaspora, (e.g., H uvarum), Hansenula, Issatchenkia, Kluyveromyces (e.g., K. phaffii), Mortierella, Mycorrhiza, Penicillium, Phycomyces, Pichia (e.g., P. anomala, P. guilliermondii, P. occidentalis, P. kudriavzevii), Pleurolus spp. (e.g., P. ostreatus), Pseudozyma (e.g., P. aphidis), Saccharomyces (e.g., S. boulardii sequela, S. cerevisiae, S. torula), Starmerella (e.g., S. bombicola), Torulopsis, Trichoderma (e.g., T. reesei, T. harzianum, T. hamatum, T. viride), Ustilago (e.g., U. maydis), Wickerhamomyces (e.g., W. anomalus), Williopsis (e.g., W. mrakii), Zygosaccharomyces (e.g., Z. bailii), and others.

In certain embodiments, the microorganism is any yeast known as a “killer yeast” characterized by its secretion of toxic proteins or glycoproteins, to which the strain itself is immune. These can include, for example, Candida (e.g., C. nodaensis), Cryptococcus, Debaryomyces (e.g., D. hansenii), Hanseniaspora, (e.g., H. uvarum), Hansenula, Kluyveromyces (e.g., K. phaffii), Pichia (e.g., P. anomala, P. guielliermondii, P. occidentalis, P. kudriavzevii), Saccharomyces (e.g., S. cerevisiae), Torulopsis, Ustilago (e.g., U. maydis), Wickerhamomyces (e.g., W. anomalus), Williopsis (e.g., W. mrakii), Zygosaccharomyces (e.g., Z bailii), and others.

In certain embodiments, the microorganisms are bacteria, including Gram-positive and Gram-negative bacteria. The bacteria may be, for example Agrobacterium (e.g., A. radiobacter), Azotobacter (A. vinelandii, A. chroococcum), Azospirillum (e.g., A. brasiliensis), Bacillus (e.g., B. amyloliquefaciens, B. circulans, B. firmus, B. laterosporus, B. lichemformis, B. megalerium, Bacillus mucilaginosus, B. subtilis), Frateuria (e.g., F. aurantia), Microbacterium (e.g., M. laevaniformans), myxobacteria (e.g., Myxococcus xanthus, Slignatella aurantiaca, Sorangium cellulosum, Minicystis rosea), Pantoea (e.g., P. agglomerans), Pseudomonas (e.g., P. aeruginosa, P. chlororaphis subsp. aureofaciens (Kluyver), P. putida), Rhizobium spp., Rhodospirillum (e.g., R. rubrum), Sphingomonas (e.g., S. paucimobilis), and/or Thiobacillus thiooxidans (Acidothiobacillus thiooxidans).

In a specific embodiment, the subject invention utilizes killer yeasts. Preferably, these yeasts are capable of colonizing a plant's roots at the root-soil interface, and providing a number of benefits to the rhizosphere. Even more specifically, the microbes of the subject invention include Wickerhamomyces anomalus (Pichia anomala). Other closely-related species are also envisioned, including other members of the Wickerhamomyces and/or Pichia clades, e.g., Pichia guilliermondii (Meyerozyma guilliermondii), Pichia kudriavzevii, and/or Pichia occidentalis.

W. anomalus has a number of beneficial characteristics useful for the present invention, including its ability to produce advantageous metabolites. For example, W. anomalus is capable of producing exo-β-1,3-glucanase, an enzyme capable of controlling or inhibiting the growth of a wide spectrum of pathogenic fungi.

In one embodiment, Wickerhamomyces anomalus is capable of producing one or more biosurfactants, including for example, a phospholipid and/or a glycolipid. In certain embodiments, the phospholipid is a cardiolipin or another phospholipid structurally-similar to cardiolipin. In certain embodiments, the glycolipid is a sophorolipid.

In addition to various by-products, this yeast is capable of producing phytase and providing a number of proteins (containing up to 50% of dry cell biomass), lipids and carbon sources, as well as a full spectrum of minerals and vitamins (B1; B2; B3 (PP); B5; B7 (H); B6; E).

In certain embodiments, the microorganism is a Trichoderma spp. fungi, such as, for example, T. harzianum, T. viride, T. hamatum, and/or T. reesei.

In addition to protecting plants from pathogens and pests, root colonization by Trichoderma spp. can enhance root growth and development, crop productivity, resistance to abiotic stresses, and bioavailability of nutrients.

In certain embodiments, the microorganism is a Bacillus spp. bacterium, such as, for example, B. subtilis and/or B. amyloliquefaciens. In one embodiment, the bacterium is B. amyloliquefaciens subsp. locus. In some embodiments, the Bacillus microbe can solubilize phosphorus compounds in the soil.

In one embodiment, the microorganism is a mycobacterium, or slime-forming bacteria. Specifically, in one embodiment, the mycobacterium is a Myxococcus spp. bacterium, e.g., M xanthus.

In certain embodiments, the microorganism is one that is capable of fixing and/or solubilizing nitrogen, potassium, phosphorous and/or other micronutrients in soil.

In one embodiment, the microorganism is a nitrogen-fixing microorganism, or a diazotroph, selected from species of, for example, Azospirillum, Azotobacter, Chlorobiaceae, Cyanothece, Frankia, Klebsiella, rhizobia, Trichodesmium, and some Archaea. In a specific embodiment, the nitrogen-fixing bacteria is Azotobacter vinelandii.

In another embodiment, the microorganism is a potassium-mobilizing microorganism, or KMB, selected from, for example, Bacillus mucilaginosus, Frateuria aurantia or Glomus mosseae. In a specific embodiment, the potassium-mobilizing microorganism is Frateuria aurantia.

In one embodiment, the combination of microorganisms applied to a plant and/or its surrounding environment is customized for a given plant and/or environment. Advantageously, in some embodiments, the combination of microbes work synergistically with one another to enhance plant health, growth and/or yields.

### Preparation of Microbe-Based Products

One microbe-based product of the subject invention is simply the fermentation medium containing the microorganisms and/or the microbial metabolites produced by the microorganisms and/or any residual nutrients. The product of fermentation may be used directly without extraction or purification. If desired, extraction and purification can be easily achieved using standard extraction and/or purification methods or techniques described in the literature.

The microorganisms in the microbe-based products may be in an active or inactive form, or in the form of vegetative cells, reproductive spores, conidia, mycelia, hyphae, or any other form of microbial propagule. The microbe-based products may also contain a combination of any of these forms of a microorganism.

In one embodiment, different strains of microbe are grown separately and then mixed together to produce the microbe-based product. The microbes can, optionally, be blended with the medium in which they are grown and dried prior to mixing.

In one embodiment, the different strains are not mixed together, but are applied to a plant and/or its environment as separate microbe-based products.

The microbe-based products may be used without further stabilization, preservation, and storage. Advantageously, direct usage of these microbe-based products preserves a high viability of the microorganisms, reduces the possibility of contamination from foreign agents and undesirable microorganisms, and maintains the activity of the by-products of microbial growth.

Upon harvesting the microbe-based composition from the growth vessels, further components can be added as the harvested product is placed into containers or otherwise transported for use. The additives can be, for example, buffers, carriers, other microbe-based compositions produced at the same or different facility, viscosity modifiers, preservatives, nutrients for microbe growth, surfactants, emulsifying agents, lubricants, solubility controlling agents, tracking agents, solvents, biocides, antibiotics, pH adjusting agents, chelators, stabilizers, ultra-violet light resistant agents, other microbes and other suitable additives that are customarily used for such preparations.

In one embodiment, buffering agents including organic and amino acids or their salts, can be added. Suitable buffers include citrate, gluconate, tartarate, malate, acetate, lactate, oxalate, aspartate, malonate, glucoheptonate, pyruvate, galactarate, glucarate, tartronate, glutamate, glycine, lysine, glutamine, methionine, cysteine, arginine and a mixture thereof. Phosphoric and phosphorous acids or their salts may also be used. Synthetic buffers are suitable to be used but it is preferable to use natural buffers such as organic and amino acids or their salts listed above.

In a further embodiment, pH adjusting agents include potassium hydroxide, ammonium hydroxide, potassium carbonate or bicarbonate, hydrochloric acid, nitric acid, sulfuric acid or a mixture. The pH of the microbe-based composition should be suitable for the microorganism(s) of interest.

In one embodiment, additional components such as an aqueous preparation of a salt, such as sodium bicarbonate or carbonate, sodium sulfate, sodium phosphate, sodium biphosphate, can be included in the formulation.

In certain embodiments, an adherent substance can be added to the composition to prolong the adherence of the product to plant parts. Polymers, such as charged polymers, or polysaccharide-based substances can be used, for example, xanthan gum, guar gum, levan, xylinan, gellan gum, curdlan, pullulan, dextran and others.

In preferred embodiments, commercial grade xanthan gum is used as the adherent. The concentration of the gum should be selected based on the content of the gum in the commercial product. If the xanthan gum is highly pure, then 0.001% (w/v—xanthan gum/solution) is sufficient.

In one embodiment, glucose, glycerol and/or glycerin can be added to the microbe-based product to serve as, for example, an osmoticum during storage and transport. In one embodiment, molasses can be included.

In one embodiment, prebiotics can be added to and/or applied concurrently with the microbe-based product to enhance microbial growth. Suitable prebiotics, include, for example, kelp extract, fulvic acid, chitin, humate and/or humic acid. In a specific embodiment, the amount of prebiotics applied is about 0.1 L/acre to about 0.5 L/acre, or about 0.2 L/acre to about 0.4 L/acre.

Optionally, the product can be stored prior to use. The storage time is preferably short. Thus, the storage time may be less than 60 days, 45 days, 30 days, 20 days, 15 days, 10 days, 7 days, 5 days, 3 days, 2 days, 1 day, or 12 hours. In a preferred embodiment, if live cells are present in the product, the product is stored at a cool temperature such as, for example, less than 20° C., 15° C., 10° C., or 5° C.

### Local Production of Microbe-Based Products

In certain embodiments of the subject invention, a microbe growth facility produces fresh, high-density microorganisms and/or microbial growth by-products of interest on a desired scale. The microbe growth facility may be located at or near the site of application. The facility produces high-density microbe-based compositions in batch, quasi-continuous, or continuous cultivation.

The microbe growth facilities of the subject invention can be located at the location where the microbe-based product will be used (e.g., a citrus grove). For example, the microbe growth facility may be less than 300, 250, 200, 150, 100, 75, 50, 25, 15, 10, 5, 3, or 1 mile from the location of use.

Because the microbe-based product can be generated locally, without resort to the microorganism stabilization, preservation, storage and transportation processes of conventional microbial production, a much higher density of microorganisms can be generated, thereby requiring a smaller volume of the microbe-based product for use in the on-site application or which allows much higher density microbial applications where necessary to achieve the desired efficacy. This allows for a scaled-down bioreactor (e.g., smaller fermentation vessel, smaller supplies of starter material, nutrients and pH control agents), which makes the system efficient and can eliminate the need to stabilize cells or separate them from their culture medium. Local generation of the microbe-based product also facilitates the inclusion of the growth medium in the product. The medium can contain agents produced during the fermentation that are particularly well-suited for local use.

Locally-produced high density, robust cultures of microbes are more effective in the field than those that have remained in the supply chain for some time. The microbe-based products of the subject invention are particularly advantageous compared to traditional products wherein cells have been separated from metabolites and nutrients present in the fermentation growth media. Reduced transportation times allow for the production and delivery of fresh batches of microbes and/or their metabolites at the time and volume as required by local demand.

The microbe growth facilities of the subject invention produce fresh, microbe-based compositions, comprising the microbes themselves, microbial metabolites, and/or other components of the medium in which the microbes are grown. If desired, the compositions can have a high density of vegetative cells or propagules, or a mixture of vegetative cells and propagules.

Advantageously, the compositions can be tailored for use at a specified location. In one embodiment, the microbe growth facility is located on, or near, a site where the microbe-based products will be used (e.g., a citrus grove).

Advantageously, these microbe growth facilities provide a solution to the current problem of relying on far-flung industrial-sized producers whose product quality suffers due to upstream processing delays, supply chain bottlenecks, improper storage, and other contingencies that inhibit the timely delivery and application of, for example, a viable, high cell-count product and the associated medium and metabolites in which the cells are originally grown.

The microbe growth facilities provide manufacturing versatility by their ability to tailor the microbe-based products to improve synergies with destination geographies. Advantageously, in preferred embodiments, the systems of the subject invention harness the power of naturally-occurring local microorganisms and their metabolic by-products to improve agricultural production.

The cultivation time for the individual vessels may be, for example, from 1 to 7 days or longer. The cultivation product can be harvested in any of a number of different ways.

Local production and delivery within, for example, 24 hours of fermentation results in pure, high cell density compositions and substantially lower shipping costs. Given the prospects for rapid advancement in the development of more effective and powerful microbial inoculants, consumers will benefit greatly from this ability to rapidly deliver microbe-based products.

### Methods of Enhancing Plant Root Health and Immune Health

In preferred embodiments, a method is provided for enhancing plant health, growth and/or yields, wherein a soil treatment composition comprising a yeast and/or a growth by-product thereof is contacted with the plant and/or its surrounding environment. Preferably, the yeast is Wickerhamomyces anomalus or a species related thereto. In some embodiments, multiple plants and/or their surrounding environments are treated according to the subject methods.

In certain embodiments, the soil treatment composition is contacted with a plant or its envrionment after the composition has been prepared, for example, by dissolving dried powder or granules in water. To improve or stabilize the effects of the treatment composition, it can be blended with suitable adjuvants and then used as such or after dilution if necessary.

In one embodiment, additional microorganisms can be applied contemporaneously with the yeast. For example, a mycobacterium such as Myxococcus xanthus can also be applied, and/or one or more microorganisms capable of fixing, mobilizing and/or solubilizing nitrogen, potassium, phosphorous (or phosphate) and/or other micronutrients in soil. In one embodiment, a nitrogen-fixing microbe, such as, for example, Azotobacter vinelandii, can also be applied. In another embodiment, a potassium-mobilizing microbe, such as, for example, Frateuria aurantia can also be applied.

In certain embodiments, the microorganisms of the composition work synergistically with one another to enhance health, growth and/or yields of plants, and/or to enhance the properties of the rhizosphere.

In some embodiments, the methods further comprise applying materials with the composition to enhance microbe growth during application (e.g., nutrients and/or prebiotics to promote microbial growth). In one embodiment, nutrient sources can include, for example, sources of nitrogen, potassium, phosphorus, magnesium, proteins, vitamins and/or carbon. In one embodiments, prebiotics can include, for example, kelp extract, fulvic acid, chitin, humate and/or humic acid.

In one embodiment, the method can enhance plant health, growth and/or yields by enhancing root health and growth. More specifically, in one embodiment, the methods can be used to improve the properties of the rhizosphere in which a plant's roots are growing, for example, the nutrient and/or moisture retention properties.

Additionally, in one embodiment, the method can be used to inoculate a rhizosphere with one or more beneficial microorganisms. For example, in preferred embodiments, the microbes of the soil treatment composition can colonize the rhizosphere and provide multiple benefits to the plant whose roots are growing therein, including protection and nourishment.

Advantageously, in one embodiment, the subject methods can be used to enhance health, growth and/or yields in plants having compromised immune health due to an infection from a pathogenic agent or from an environmental stressor, such as, for example, drought. Thus, in certain embodiments, the subject methods can also be used for improving the immune health, or immune response, of plants.

As used herein, “applying” a composition or product refers to contacting a composition or product with a target or site such that the composition or product can have an effect on that target or site. The effect can be due to, for example, microbial growth and/or interaction with a plant, as well as the action of a metabolite, enzyme, biosurfactant or other microbial growth by-product. Applying can also include “treating” a target or site with a composition.

Application can further include contacting the microbe-based product directly with a plant, plant part, and/or the plant's surrounding environment (e.g., the soil or the rhizosphere). As used herein, a plant's “surrounding environment” means the soil and/or other medium in which the plant is growing, which can include the rhizosphere. In certain embodiments, the surrounding environment does not extend past, for example, a radius of at least 5 miles, 1 mile, 1,000 feet, 500 feet, 300 feet, 100 feet, 10 feet, 8 feet, or 6 feet from the plant.

The microbe-product can be applied as a seed treatment or to the soil surface, or to the surface of a plant or plant part (e.g., to the surface of the roots, tubers, stems, flowers, leaves, fruit, or flowers). It can be sprayed, poured, sprinkled, injected or spread as liquid, dry powder, dust, granules, microgranules, pellets, wettable powder, flowable powder, emulsions, microcapsules, oils, gels, pastes or aerosols.

In a specific embodiment, the composition is contacted with one or more roots of the plant. The composition can be applied directly to the roots, e.g., by spraying or dunking the roots, and/or indirectly, e.g., by administering the composition to the soil in which the plant grows (e.g., the rhizosphere). The composition can be applied to the seeds of the plant prior to or at the time of planting, or to any other part of the plant and/or its surrounding environment.

In certain embodiments, the compositions provided herein are applied to the soil surface without mechanical incorporation. The beneficial effect of the soil application can be activated by rainfall, sprinkler, flood, or drip irrigation, and subsequently delivered to, for example, the roots of plants.

Plants and/or their environments can be treated at any point during the process of cultivating the plant. For example, the soil treatment composition can be applied to the soil prior to, concurrently with, or after the time when seeds are planted therein. It can also be applied at any point thereafter during the development and growth of the plant, including when the plant is flowering, fruiting, and during and/or after abscission of leaves.

In one embodiment, the method can be used in a large scale agricultural setting. The method can comprise administering the soil treatment composition into a tank connected to an irrigation system used for supplying water, fertilizers or other liquid compositions to a crop, orchard or field. Thus, the plant and/or soil surrounding the plant can be treated with the soil treatment composition via, for example, soil injection, soil drenching, or using a center pivot irrigation system, or with a spray over the seed furrow, or with sprinklers or drip irrigators. Advantageously, the method is suitable for treating hundreds of acres of crops, orchards or fields at one time.

In one embodiment, the method can be used in a smaller scale setting, such as in a home garden or greenhouse. In such cases, the method can comprise spraying a plant and/or its surrounding environment with the soil treatment composition using a handheld lawn and garden sprayer. The composition can be mixed with water, and optionally, other lawn and garden treatments, such as fertilizers and pesticides. The composition can also be mixed in a standard handheld watering can and poured onto soil.

In certain embodiments, the plant receiving treatment is healthy. Advantageously, the subject invention can be useful in enhancing the immune response of a plant having a compromised immune system, for example, because the plant is affected by disease and/or disease symptoms.

For example, the plant may be affected by a pathogenic strain of Pseudomonas (e.g., P. savastanoi, P. syringae pathovars); Ralstonia solanacearum; Agrobacterium (e.g., A. tumefaciens); Xanthomonas (e.g., X. oryzae pv. Oryzae, X. campestris pathovars. X. axonopodis pathovars); Erwinia (e.g., E. amylovora); Xylella (e.g., X. fastidiosa); Dickeya (e.g., D. dadantii and D. solani); Pectobacterium (e.g., P. carotovorum and P. atrosepticum); Clavibacter (e.g., C. michiganensis and C. sepedonicus); Candidatus Liberibacter asiaticus; Pantoea; Burkholderia; Acidovorax; Streptomyces; Spiroplasma; and/or Phytoplasma; as well as huanglongbing (HLB, citrus greening disease), citrus canker disease, citrus bacterial spot disease, citrus variegated chlorosis, brown rot, citrus root rot, citrus and black spot disease.

In one embodiment, the methods are used to enhance the health, growth and/or yields of citrus trees affected by citrus greening disease and/or citrus canker disease.

The present invention can be used to enhance health, growth and/or yields of plants and/or crops in, for example, agriculture, horticulture, greenhouses, landscaping, and the like. The present invention can also be used for improving one or more qualities of soil, thereby enhancing the performance of the soils for agricultural, home and gardening purposes. Furthermore, the present invention can be used in pasture management, as well as in professional turf and landscape management.

In certain embodiments, the soil treatment composition may also be applied so as to promote colonization of the roots and/or rhizosphere as well as the vascular system of the plant in order to enhance plant health and vitality. Thus, growth of nutrient-fixing microbes such as Rhizobium and/or Mycorrhizae can be promoted, as well as other beneficial endogenous and exogenous microbes, and/or their by-products that promote crop growth, health and/or yield. The microbe-based product can also support a plant's vascular system by, for example, entering and colonizing said vascular system and contributing metabolites, and nutrients important to plant health and productivity.

In yet another embodiment, the method can be used to fight off and/or discourage colonization of the rhizosphere by soil microorganisms that are deleterious or that might compete with beneficial soil microorganisms.

In one embodiment, the method can be used for enhancing penetration of beneficial molecules through the outer layers of root cells, for example, at the root-soil interface of the rhizosphere.

The subject invention can be used to improve any number of qualities of any type of soil, for example, clay, sandy, silty, peaty, chalky, loam soil, and/or combinations thereof. Furthermore, the methods and compositions can be used for improving the quality of dry, waterlogged, porous, depleted, compacted soils and/or combinations thereof. Soil can include the soil present in the rhizosphere or soil that lies outside of the rhizosphere.

In one embodiment, the method can be used for improving the drainage and/or dispersal of water in waterlogged soils. In one embodiment, the method can be used for improving water retention in dry soil.

In one embodiment, the method can be used for improving nutrient retention in porous and/or depleted soils. Furthermore, in one embodiment, the method can be used to provide a nutrient to a plant, and/or to treat and/or prevent a nutrient deficiency in a plant. For example, in one embodiment, when the yeasts of the soil treatment composition die, their cells provide an abundance of nutrients, proteins, vitamins, and minerals for the plants and or for other soil microbiota to utilize.

In another embodiment, wherein the yeast of the soil treatment composition produces phytase, the method can be used to provide the plant with phosphorus in the form of phosphates. Phytase is capable of converting phytic acid present in soil into plant-bioavailable (e.g., root-absorbable) phosphates. Accordingly, the method can be used to treat and/or prevent a phosphorus deficiency in a plant.

In one embodiment, the method controls pathogenic bacteria. In one embodiment, the method works to indirectly enhance plant immune responses by enhancing the immune health of plants and increase the ability to fight off infections.

In yet another embodiment, the method controls pests that might act as vectors or carriers for pathogenic bacteria. In most plant diseases caused by plant pathogenic bacteria (especially in those that cause spots, cankers, blights, galls, or soft rots), the bacteria can escape to the surface of their host plants as droplets or masses of sticky exudates. The bacterial exudates are released through cracks or wounds in the infected area, or through natural openings in the infected area of the plant. Such bacteria are then likely to stick on the legs and bodies of insects, such as flies, aphids, ants, beetles, whiteflies, etc., that land on the plant and come in contact with the substance.

Many of these insects are attracted by sugars contained in the bacterial exudate, which they feed on and further smear onto their body and mouthparts. When the insects move to other parts of the plant or to other susceptible host plants, they carry numerous bacteria on their body. If the insects happen to land on a fresh wound or on a natural opening in a plant, and there is enough moisture on the plant surface, the bacteria may multiply, move into the plant, and begin a new infection. Thus, the subject methods can prevent the spread of plant pathogenic bacteria by controlling, e.g., killing, these carrier pests.

The microbe-based products can be used either alone or in combination with other compounds for efficient enhancement of plant health, growth and/or yields, as well as other compounds for efficient treatment and prevention of plant pathogenic pests. For example, the methods can be used concurrently with sources of nutrients and/or micronutrients for enhancing plant and/or microbe growth, such as magnesium, phosphate, nitrogen, potassium, selenium, calcium, sulfur, iron, copper, and zinc; and/or one or more prebiotics, such as kelp extract, fulvic acid, chitin, humate and/or humic acid. The exact materials and the quantities thereof can be determined by a grower or an agricultural scientist having the benefit of the subject disclosure.

The compositions can also be used in combination with other agricultural compounds and/or crop management systems. In one embodiment, the composition can optionally comprise, and/or be applied with, for example, natural and/or chemical pesticides, repellants, herbicides, fertilizers, water treatments, non-ionic surfactants and/or soil amendments.

In one embodiment, the subject compositions can be used with agricultural compounds characterized as antiscalants, such as, e.g., hydroxyethylidene diphosphonic acid;

bactericides, such as, e.g., streptomycin sulfate and/or Galltrol® (A. radiobacter strain K84);

biocides, such as, e.g., chlorine dioxide, didecyldimethyl ammonium chloride, halogenated heterocyclic, and/or hydrogen dioxide/peroxyacetic acid;

fertilizers, such as, e.g., N-P-K fertilizers, calcium ammonium nitrate 17-0-0, potassium thiosulfate, nitrogen (e.g., 10-34-0, Kugler KQ-XRN, Kugler KS-178C, Kugler KS-2075, Kugler LS 6-24-6S, UN 28. UN 32), and/or potassium;

fungicides, such as, e.g., chlorothalonil, manicozeb hexamethylenetetramine, aluminum tris, azoxystrobin, Bacillus spp. (e.g., B. licheniformis strain 3086, B. subtilis, B. subtilis strain QST 713), benomyl, boscalid, pyraclostrobin, captan, carboxin, chloroneb, chlorothalonil, copper culfate, cyazofamid, dicloran, dimethomorph, etridiazole, thiophanate-methyl, fenamidone, fenarimol, fludioxonil, fluopicolide, flutolaniL iprodione, mancozeb, maneb, mefanoxam. Hudioxonil, mefenoxam, metalaxyl, myclobutanil, oxathiapiprolin, pentachloronitrobenzene (quintozene), phosphorus acid, propamocarb, propanil, pyraclostrobin, Reynoutria sachalinensis, Streptomyces spp. (e.g., S. griseoviridis strain K61, S. lydicus WYEC 108), sulfur, urea, thiabendazole, thiophanate methyl, thiram, triadimefon, triadimenol, and/or vinclozolin;

growth regulators, such as, e.g., ancymidol, chlormequat chloride, diaminozide, paclobutrazol, and/or uniconazole;

herbicides, such as, e.g., glyphosate, oxyfluorfen, and/or pendimethalin;

insecticides, such as, e.g., acephate, azadirachtin, B. thuringiensis (e.g., subsp. israelensis strain AM 65-52), Beauveria bassiana (e.g., strain GIHA), carbaryl, chlorpyrifos, cyantraniliprole, cyromazine, dicofol, diazinon, dinotefuran, imidaeloprid, Isariafiimosorosae (e.g., Apopka strain 97), lindane, and/or malathion;

water treatments, such as, e.g., hydrogen peroxide (30-35%), phosphonic acid (5-20%), and/or sodium chlorite;

as well as glycolipids, lipopeptides, deet, diatomaceous earth, citronella, essential oils, mineral oils, garlic extract, chili extract, and/or any known commercial and/or homemade pesticide that is determined to be compatible by the skilled artisan having the benefit of the subject disclosure.

In certain embodiments, the microbe-based products can be used to enhance the effectiveness of the other compounds, for example, by enhancing the penetration of a drug compound into a plant or pest. The microbe-based products can also be used to supplement other treatments, for example, antibiotic treatments. Advantageously, the subject invention helps reduce the amount of antibiotics that must be administered to a crop or plant in order to be effective at treating and/or preventing bacterial infection.

In one embodiment, the methods and compositions according to the subject invention lead to an increase in one or more of: growth index, root mass, plant height, trunk diameter, shoot growth, shoot count, canopy density, brix value, chlorophyll content, fruit count, fruit mass, root mass, total plant biomass, flower count and/or leaf tissue nitrogen levels of a plant, by about 5%, 10%, 20%, 30%, 40%, 50%, 60% 70%, 80%, 90%, 100%, 150%, 200%, or more, compared to a plant growing in an untreated environment.

In certain embodiments, the methods and compositions according to the subject invention lead to an increase in crop yield by about 5%, 10%, 20%, 30%, 40%, 50%, 60% 70%, 80%, 90%, 100%, 150%, 200%, or more, compared to untreated crops.

In one embodiment, the methods and compositions according to the subject invention lead to a reduction in the number of pests on a plant or in a plant's surrounding environment by about 55%, 10%, 20%, 30%, 40%, 50%, 60% 70%, 80%, 90%, 100%, 150%, 200%, or more, compared to a plant growing in an untreated environment.

In one embodiment, the methods and compositions according to the subject invention reduce damage to a plant caused by pests by about 5%, 10%, 20%, 30%, 40%, 50%, 60% 70%, 80%, 90%, 100%, 150%, 200%, or more, compared to plants growing in an untreated environment.

### Target Plants

As used here, the term “plant” includes, but is not limited to, any species of woody, ornamental or decorative, crop or cereal, fruit plant or vegetable plant, flower or tree, macroalga or microalga, phytoplankton and photosynthetic algae (e.g., green algae Chlamydomonas reinhardtii). “Plant” also includes a unicellular plant (e.g. microalga) and a plurality of plant cells that are largely differentiated into a colony (e.g. volvox) or a structure that is present at any stage of a plant's development. Such structures include, but are not limited to, a fruit, a seed, a shoot, a stem, a leaf, a root, a flower petal, etc. Plants can be standing alone, for example, in a garden, or can be one of many plants, for example, as part of an orchard, crop or pasture.

As used herein, “crop plants” refer to any species of plant or alga edible by humans or used as a feed for animals or fish or marine animals, or consumed by humans, or used by humans (e.g., textile or cosmetics production), or viewed by humans (e.g., flowers or shrubs in landscaping or gardens) or any plant or alga, or a part thereof, used in industry or commerce or education.

Types of crop plants that can benefit from application of the products and methods of the subject invention include, but are not limited to: row crops (e.g., corn, soy, sorghum, peanuts, potatoes, etc.), field crops (e.g., alfalfa, wheat, grains, etc.), tree crops (e.g., walnuts, almonds, pecans, hazelnuts, pistachios, etc.), citrus crops (e.g., orange, lemon, grapefruit, etc.), fruit crops (e.g., apples, pears, strawberries, blueberries, blackberries, etc.), turf crops (e.g., sod), ornamentals crops (e.g., flowers, vines, etc.), vegetables (e.g., tomatoes, carrots, etc.), vine crops (e.g., grapes, etc.), forestry (e.g., pine, spruce, eucalyptus, poplar, etc.), managed pastures (any mix of plants used to support grazing animals).

Additional examples of plants for which the subject invention is useful include, but are not limited to, cereals and grasses (e.g., wheat, barley, rye, oats, rice, maize, sorghum, corn), beets (e.g., sugar or fodder beets); fruit (e.g., grapes, strawberries, raspberries, blackberries, pomaceous fruit, stone fruit, soft fruit, apples, pears, plums, peaches, almonds, cherries or berries); leguminous crops (e.g., beans, lentils, peas or soya); oil crops (e.g., oilseed rape, mustard, poppies, olives, sunflowers, coconut, castor, cocoa or ground nuts); cucurbits (e.g., pumpkins, cucumbers, squash or melons); fiber plants (e.g., cotton, flax, hemp or jute); citrus fruit (e.g., oranges, lemons, grapefruit or tangerines); vegetables (e.g., spinach, lettuce, asparagus, cabbages, carrots, onions, tomatoes, potatoes or bell peppers); Lauraceae (e.g., avocado, Cinnamonium or camphor); and also tobacco, nuts, herbs, spices, medicinal plants, coffee, eggplants, sugarcane, tea, pepper, grapevines, hops, the plantain family, latex plants, cut flowers and ornamentals.

In certain embodiments, the crop plant is a citrus plant. Examples of citrus plants according to the subject invention include, but are not limited to, orange trees, lemon trees, lime trees and grapefruit trees. Other examples include Citrus maxima (Pomelo), Citrus medica (Citron), Citrus micrantha (Papeda), Citrus reticulata (Mandarin orange), Citrus paradisi (grapefruit), Citrus japonica (kumquat), Citrus australasica (Australian Finger Lime), Citrus australis (Australian Round lime), Citrus glauca (Australian Desert Lime), Citrus garrawayae (Mount White Lime), Citrus gracilis (Kakadu Lime or Humpty Doo Lime), Citrus inodora (Russel River Lime), Citrus warburgiana (New Guinea Wild Lime), Citrus wintersii (Brown River Finger Lime), Citrus halimii (limau kadangsa, limau kedut kera), Citrus indica (Indian wild orange), Citrus macroptera, and Citrus latipes, Citrus x aurantiifolia (Key lime), Citrus x aurantium (Bitter orange), Citrus x latifolia (Persian lime), Citrus x limon (Lemon), Citrus x limonia (Rangpur), Citrus x sinensis (Sweet orange), Citrus x tangerina (Tangerine), Imperial lemon, tangelo, orangelo, tangor, kinnow, kiyomi, Minneola tangelo, oroblanco, ugli, Buddha's hand, citron, bergamot orange, blood orange, calamondin, clementine, Meyer lemon, and yuzu.

In some embodiments, the crop plant is a relative of a citrus plant, such as orange jasmine, limeberry, and trifoliate orange (Citrus trifolata).

Additional examples of target plants include all plants that belong to the superfamily Viridiplantae, in particular monocotyledonous and dicotyledonous plants including fodder or forage legumes, ornamental plants, food crops, trees or shrubs selected from Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp, Artocarpus spp., Asparagus officinalis, Avena spp. (e.g., A. sativa, A. fatua, A. byzantina, A. fatua var. sativa, A. hybrida), Averrhoa carambola, Bambusa sp., Benincasa hispida, Bertholletia excelsea, Beta vulgaris, Brassica spp. (e.g., B. napus, B. rapa ssp. [canola, oilseed rape, turnip rape]), Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g., E. guineensis, E. oleifera), Eleusine coracana, Eragrostis tef, Erianthus sp., Eriobotrya japonica, Eucalyptus sp., Eugenia uniora, Fagopyrum spp., Fagus spp., Festuca arundinacea, Ficus carica, Fortunella spp., Fragaria spp., Ginkgo biloba, Glycine spp. (e.g., G. max, Soja hispida or Soja max), Gossypium hirsutum, Helianthus spp. (e.g., H. annuus), Hemerocallis fulva, Hibiscus spp., Hordeum spp. (e.g., H. vulgare), Ipomoea batatas, Juglans spp., Lactuca saliva, Lathyrus spp., Lens culinaris, Linum usitatissimum, Litchi chinensis, Lotus spp., Luffa acutangula, Lupinus spp., Luzula sylvatica, Lycopersicon spp. (e.g., L. esculentum, L. lycopersicum, L. pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g., O. saliva, O. latifolia), Panicum miliaceum, Panicum virgatum, Passiflora edulis, Pastinaca saliva, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp. (e.g., S. tuberosum, S. integrifolium or S. lycopersicum), Sorghum bicolor, Spinacia spp., Syzygium spp., Tagetes spp., Tamarindus indica, Theobroma cacao, Trifolium spp., Tripsacum dactyloides, Triticosecale rimpaui, Triticum spp. (e.g., T. aestivum, T. durum, T. turgidum, T. hybernum, T. macha, T. sativum, T. monococcum or T. vulgare), Tropaeolum minus, Tropaeolum majus, Vaccinium spp., Vicia spp., Vigna spp., Viola odorata, Vitis spp., Zea mays, Zizania palustris, Ziziphus spp., amongst others.

Target plants can also include, but are not limited to, corn (Zea mays), Brassica sp. (e.g., B. napus, B. rapa, B. juncea), particularly those Brassica species useful as sources of seed oil, alfalfa (Medicago saliva), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

Target vegetable plants include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum. Conifers that may be employed in practicing the embodiments include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis). Plants of the embodiments include crop plants (for example, corn, alfalfa, sunflower, Brassica, soybean, cotton, safflower, peanut, sorghum, wheat, millet, tobacco, etc.), such as corn and soybean plants.

Target turfgrasses include, but are not limited to: annual bluegrass (Poa annua); annual ryegrass (Lolium multiflorum); Canada bluegrass (Poa compressa); Chewings fescue (Festuca rubra); colonial bentgrass (Agrostis tenuis); creeping bentgrass (Agrostis palustris); crested wheatgrass (Agropyron desertorum); fairway wheatgrass (Agropyron cristatum); hard fescue (Festuca longifolia); Kentucky bluegrass (Poa pratensis); orchardgrass (Dactylis glomerate); perennial ryegrass (Lolium perenne); red fescue (Festuca rubra); redtop (Agrostis alba); rough bluegrass (Poa trivialis); sheep fescue (Festuca ovine); smooth bromegrass (Bromus inermis); tall fescue (Festuca arundinacea); timothy (Phleum pretense); velvet bentgrass (Agrostis canine); weeping alkaligrass (Puccinellia distans); western wheatgrass (Agropyron smithii); Bermuda grass (Cynodon spp.); St. Augustine grass (Stenotaphrum secundatum); zoysia grass (Zoysia spp.); Bahia grass (Paspalum notatum); carpet grass (Axonopus affinis); centipede grass (Eremochloa ophiuroides); kikuyu grass (Pennisetum clandesinum); seashore paspalum (Paspalum vaginatum); blue gramma (Bouteloua gracilis); buffalo grass (Buchloe dactyloids); sideoats gramma (Bouteloua curtipendula).

Further plants of interest include grain plants that provide seeds of interest, oil-seed plants, and leguminous plants. Seeds of interest include grain seeds, such as corn, wheat, barley, rice, sorghum, rye, millet, etc. Oil-seed plants include cotton, soybean, safflower, sunflower, Brassica, maize, alfalfa, palm, coconut, flax, castor, olive etc. Leguminous plants include beans and peas. Beans include guar, locust bean, fenugreek, soybean, garden beans, cowpea, mungbean, lima bean, fava bean, lentils, chickpea, etc.

Further plants of interest include Cannabis (e.g., sativa, indica, and ruderalis) and industrial hemp.

All plants and plant parts can be treated in accordance with the invention. In this context, plants are understood as meaning all plants and plant populations such as desired and undesired wild plants or crop plants (including naturally occurring crop plants). Crop plants can be plants that can be obtained by traditional breeding and optimization methods or by biotechnological and recombinant methods, or combinations of these methods, including the transgenic plants and the plant varieties.

Plant parts are understood as meaning all aerial and subterranean parts and organs of the plants such as shoot, leaf, flower and root, examples which may be mentioned being leaves, needles, stalks, stems, flowers, fruit bodies, fruits and seeds, but also roots, tubers and rhizomes. The plant parts also include crop material and vegetative and generative propagation material, for example cuttings, tubers, rhizomes, slips and seeds.

In some embodiments, the plant is a plant infected by a pathogenic disease or pest. In specific embodiments, the plant is infected with citrus greening disease and/or citrus canker disease, and/or a pest that carries such diseases.

### EXAMPLES

A greater understanding of the present invention and of its many advantages may be had from the following examples, given by way of illustration. The following examples are illustrative of some of the methods, applications, embodiments and variants of the present invention. They are not to be considered as limiting the invention. Numerous changes and modifications can be made with respect to the invention.

### Example 1—Solid State Fermentation of Wickerhamomyces anomalus

For Wickerhamomyces sp. biomass production, a rice-based medium is used. Approximately 200 grams of rice is mixed with 600 ml of GUY medium (glucose, urea, and yeast extract, pH 5.71) or 250 ml of concentrated GY medium (glucose and yeast extract, pH 5.69), and water. The media is spread onto stainless steel pans in a layer about 1 to 2 inches think, and sterilized.

Following sterilization, the pans are inoculated with seed culture. Optionally, added nutrients can be included to enhance microbial growth, including, for example, salts and/or carbon sources such as molasses, starches, glucose and sucrose.

Seed culture of Wickerhamomyces is then sprayed or pipetted onto the surface of the substrate and the trays are incubated between 28-30° C. in an enclosed reactor. Ambient air is pumped through the reactor to stabilize the temperature. Incubation for 48-72 hours can produce 1×10⁹ cells/gram or more of Wickerhamomyces.

### Example 2—Preparation of Microbe-Based Product

A sealable pouch can be used to store and transport a product comprising a product containing at least 1×10⁸ CFU/ml of Wickerhamomyces anomalus blended with residual microbial fermentation broth (e.g., 10.0% microbial inoculant and 90% broth by volume). Other components can be added to the product, e.g., micronutrients, macronutrients, prebiotics and/or other microbes similarly produced.

The product is then diluted with water in a mixing tank to a concentration of 1×10⁶ to 1×10⁷ CFU/ml. One bag can be used to treat approximately 1-10 acres of crop or citrus grove.

The composition can be mixed with and/or applied concurrently with additional “starter” materials to promote initial growth of the microorganisms in the composition. These can include, for example, prebiotics and/or nano-fertilizers (e.g., Aqua-Yield, NanoGro™).

One exemplary formulation of a starter composition comprises:
Soluble potash (K2O) (1.0% to 2.5%, or about 2.0%)
Magnesium (Mg) (0.25% to 0.75%, or about 0.5%)
Sulfur (S) (2.5% to 3.0%, or about 2.7%)
Boron (B) (0.01% to 0.05%, or about 0.02%)
Iron (Fe) (0.25% to 0.75%, or about 0.5%)
Manganese (Mn) (0.25% to 0.75%, or about 0.5%)
Zinc (Zn) (0.25% to 0.75%, or about 0.5%)
Humic acid (8% to 12%, or about 10%)
Kelp extract (5% to 10%, or about 6%)
Water (70% to 85%, or about 77% to 80%).

The microbial inoculant, and/or optional growth-promoting “starter” materials, are mixed with water in an irrigation system tank and applied to soil.

### Example 3—Young Citrus Tree Field Study in Florida

Growth response of young citrus trees in Florida was evaluated after treatment with a composition according to embodiments of the subject invention comprising W. anomalus (“STR10”). Trunk caliper (diameter), height and growth index were measured and compared to control trees grown using standard grower's practice. These factors are important factors for growers to evaluate the vigor of newly planted, non-bearing citrus trees. More vigorous growth means that the trees are healthier and will begin producing fruit crop sooner. This can be advantageous, as the process for citrus trees to reach maturity can take five years or more.

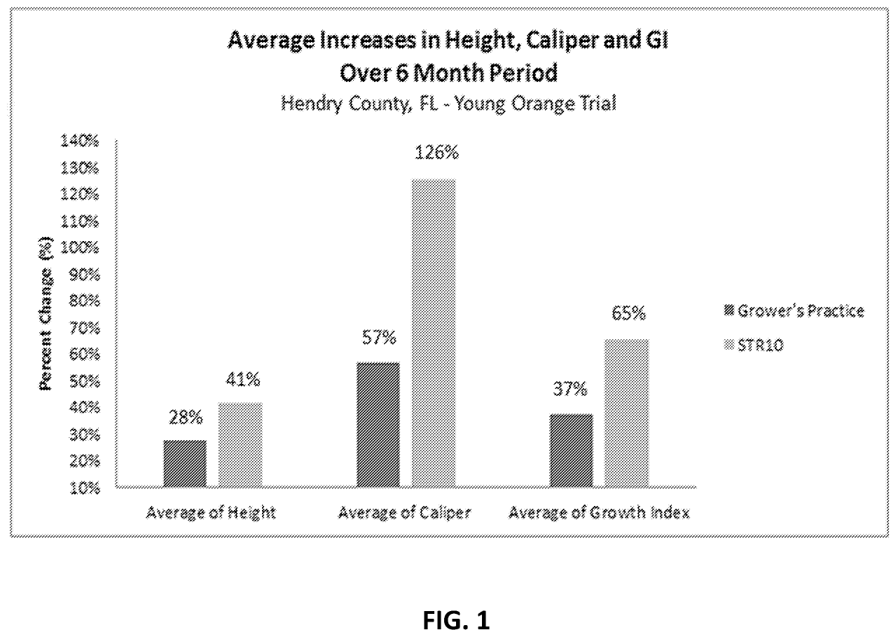

The results of the study, depicted in FIG. 1, showed that, over the course of a 6 month period, young trees treated with STR10 experienced 41% increase in average height, 126% increase in average caliper measurement, and 65% increase in growth index. The control experienced a 28% increase in average height, 57% increase in average trunk caliper, and 37% increase in average growth index.

### Example 4—Young Citrus Tree Field Studies in Florida

Growth response of young citrus trees in Florida was evaluated after treatment with a composition according to embodiments of the subject invention comprising W. anomalus (“STR10”).

### Height

The composition was applied to the soil bi-monthly for seven total treatments at 1.5 L of composition per acre.

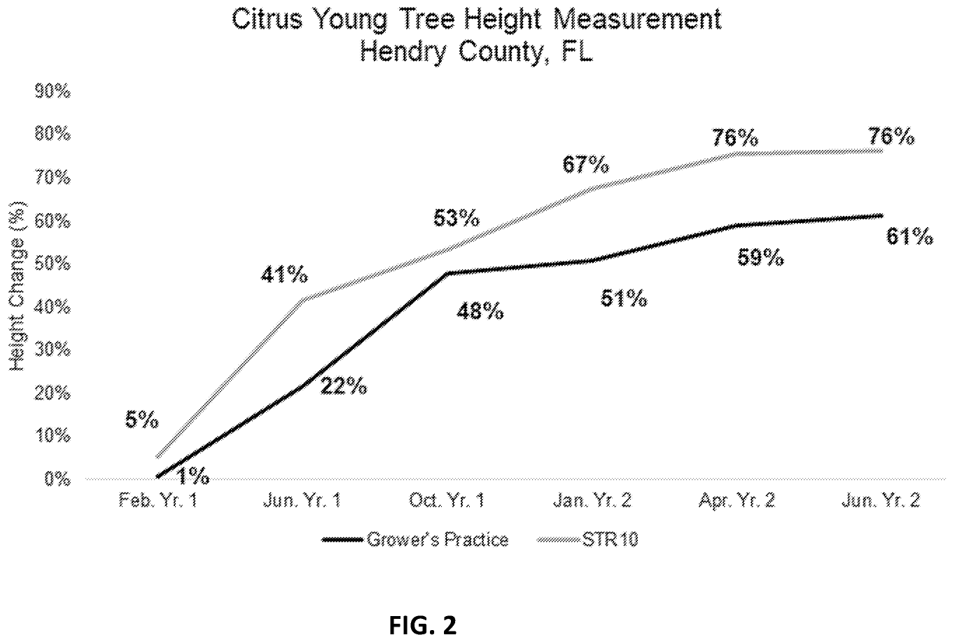

Tree height was measured and compared to control trees grown using standard grower's practice. The results of the study, depicted in FIG. 2, showed that, over the course of an 18-month period, young trees treated with the subject composition experienced a total increase in average height that was 15% greater than the height increase of the control (76% increase, treated versus 61% increase, control).

### Growth Index

The composition was applied to the soil bi-monthly for seven total treatments at 1.5 L of composition per acre.

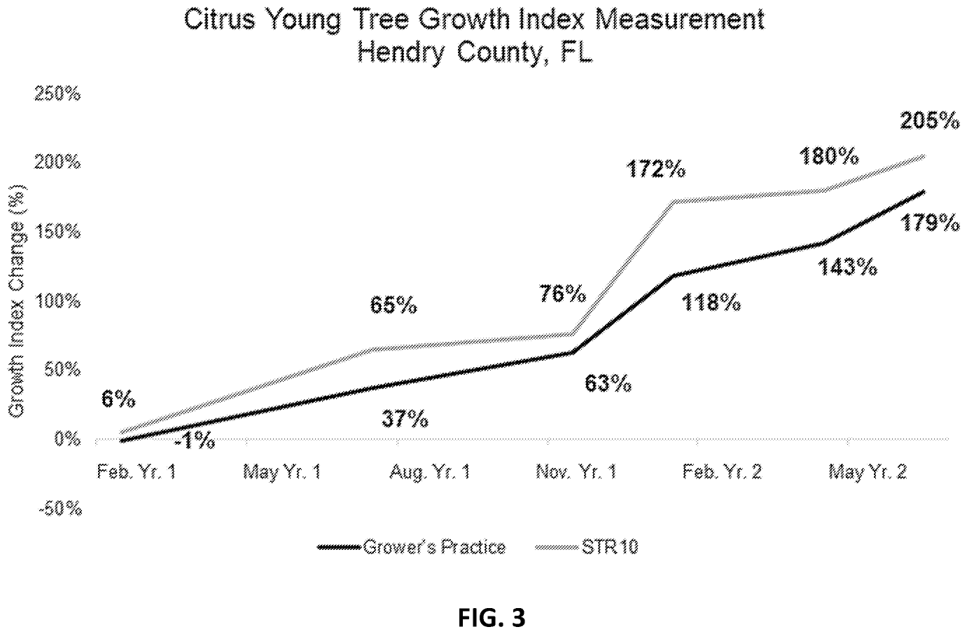

Growth Index (GI) was measured and compared to control trees grown using standard grower's practice. The results of the study, depicted in FIG. 3, showed that, over the course of an 18-month period, young trees treated with the subject composition experienced a total increase in GI that was 26% greater than the GI increase of the control (205% increase, treated versus 179% increase, control).

### Caliper

The composition was applied to the soil bi-monthly for seven total treatments at 1.5 L of composition per acre.

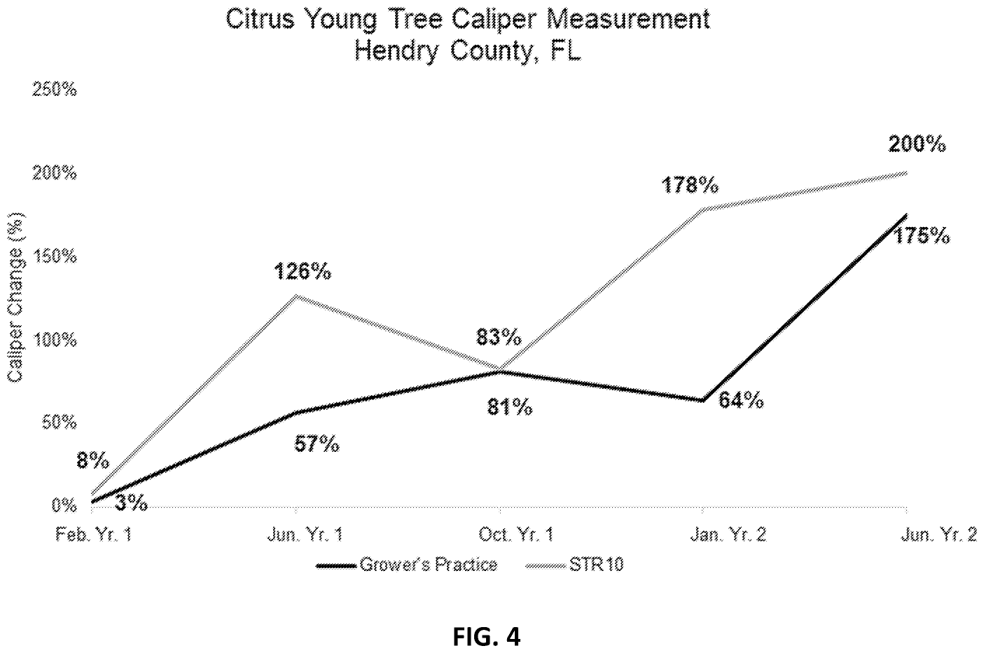

Trunk caliper was measured and compared to control trees grown using standard grower's practice. The results of the study, depicted in FIG. 4, showed that, over the course of an 18-month period, young trees treated with the subject composition experienced a total increase in caliper that was 25% greater than the caliper increase of the control (200% increase, treated versus 175% increase, control).

### Shoot Growth

The composition was applied to the soil bi-monthly for four total treatments at 1.5 L of composition per acre.

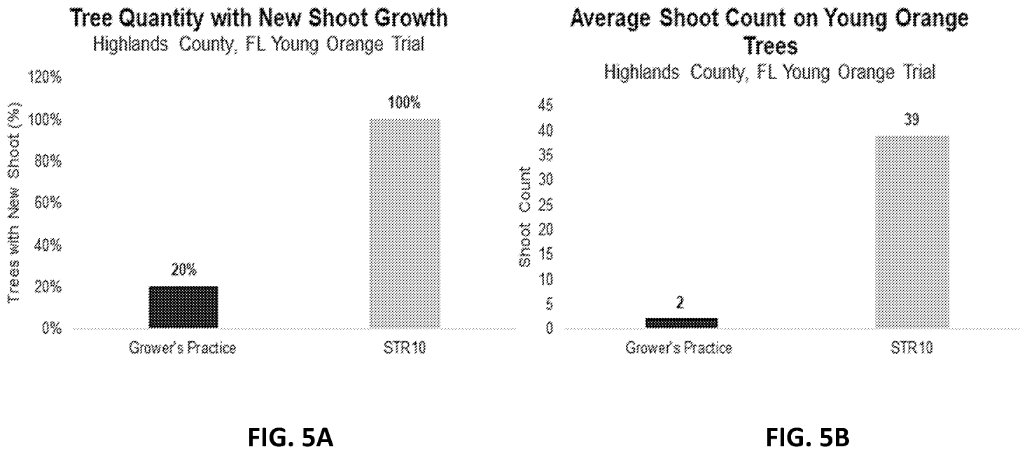

The number of trees with new shoot growth was measured and compared to control trees grown using standard grower's practice. The results of the study, depicted in FIG. 5A, showed that 100% of the treated trees had new shoot growth, whereas only 20% of the control trees did.

The average shoot count of treated trees versus untreated control tree was also measured. The results of the study, depicted in FIG. 5B, showed an average of 39 new shoots on the treated trees versus an average of 2 for control trees.

### Fruit Count Per Tree

The composition was applied to the soil bi-monthly for six total treatments at 1.5 L of composition per acre.

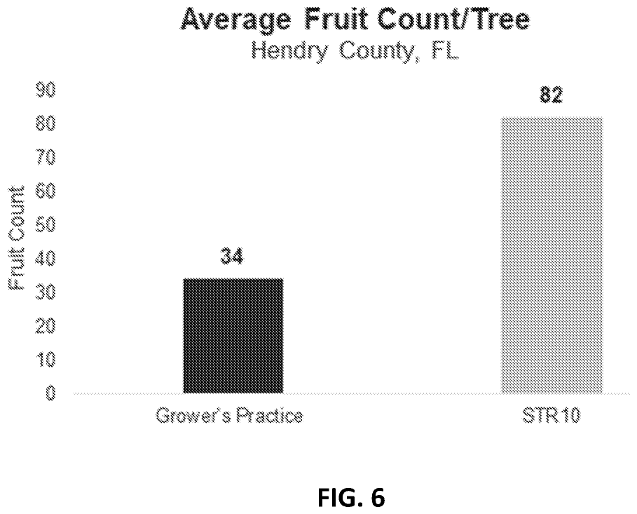

The average fruit count was measured and compared to control trees grown using standard grower's practice. The results of the study, depicted in FIG. 6, showed an average fruit count of 82 fruits per tree for the treated trees, versus an average fruit count of 34 fruits per control tree.

### Example 5—STR10+Additional Microorganisms for Lettuce

### Trial 1

A composition comprising Wickerhamomyces anomalus (“STR10”) was applied to soil in which Buttercrunch lettuce was planted and the average lettuce head weight was compared to untreated control lettuce. The composition was applied bi-weekly for a total of 3 applications, at 34 fl. oz./acre.

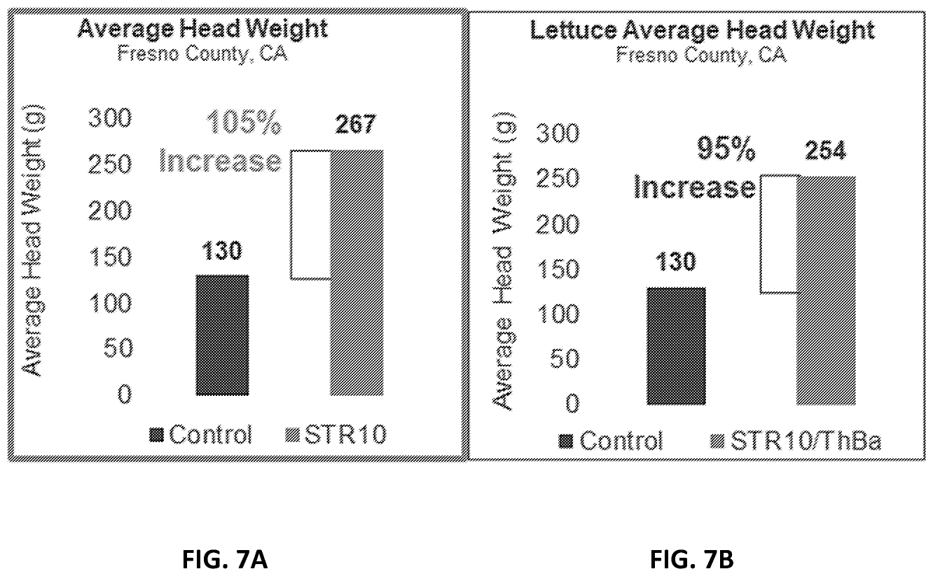

As depicted in FIG. 7A, the treated lettuce exhibited a 105% greater average head weight (g) over the control lettuce.

### Trial 2

Compositions according to embodiments of the subject invention were applied to soil in which Buttercrunch lettuce was planted, bi-weekly for a total of three treatments, and compared to untreated control lettuce. The first two treatments comprised 34 fl. oz./acre of a composition comprising Wickerhamomyces anomalus (“STR10”). The third treatment comprised 3 fl. oz./acre of a composition comprising Trichoderma harzianum and Bacillus amyloliquefaciens (“ThBa”).

As depicted in FIG. 7B, the treated lettuce exhibited a 95% greater average head weight (g) over the control lettuce.

### Example 6—STR10+Starter Materials for Georgia Peanuts

5 fl. oz./acre of a composition comprising Wickerhamomyces anomalus (“STR10”) was applied once at planting to a peanut plant plot, along with 6.4 fl. oz./acre of a “starter” composition as described in Example 2 supra. Preferably, the starter composition comprises, at least, humic acid and kelp extract. The treated peanut plants were compared with untreated controls in terms of average flower count per 30 sq. ft., and average canopy size (in.).

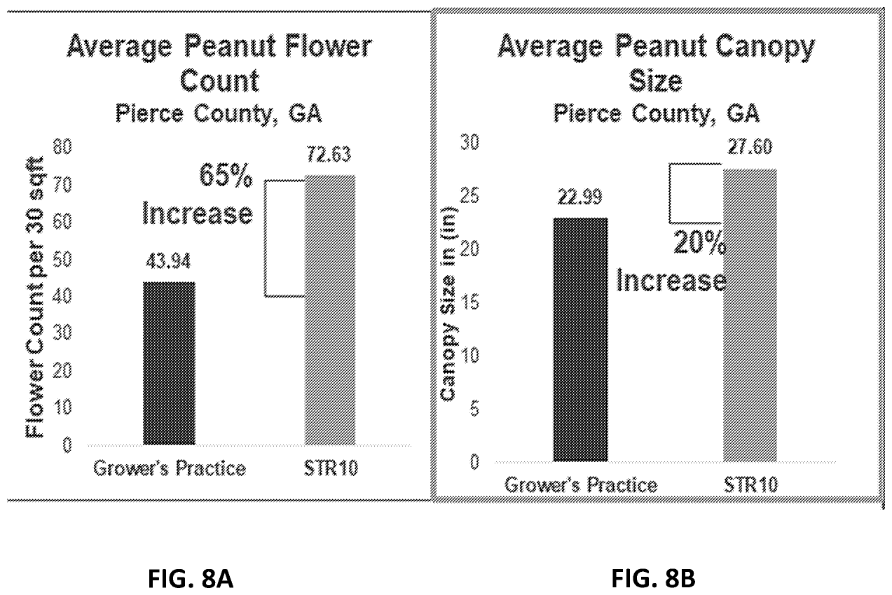

As depicted in FIG. 8A, the treated peanut plants exhibited a 65% greater flower count than the control plants. Additionally, as depicted in FIG. 8B, the treated peanut plants exhibited a 20% greater canopy size than the control plants.

### Example 7—Nutrient Uptake—Zucchini

Three treatment groups of zucchini plants were carried out. A first treatment comprised applying 5 fl. oz./acre of Wickerhamomyces anomalus (“STR10”) to the plants initially, and then once more after three weeks. The second treatment comprised applying 5 fl. oz./acre of STR10 plus 3 fl. oz./acre of a composition comprising Trichoderma harzianum and Bacillus amyloliquefaciens (“ThBa”) to the plants initially, and then once more after three weeks. The third treatment was a control, untreated group of zucchini plants.

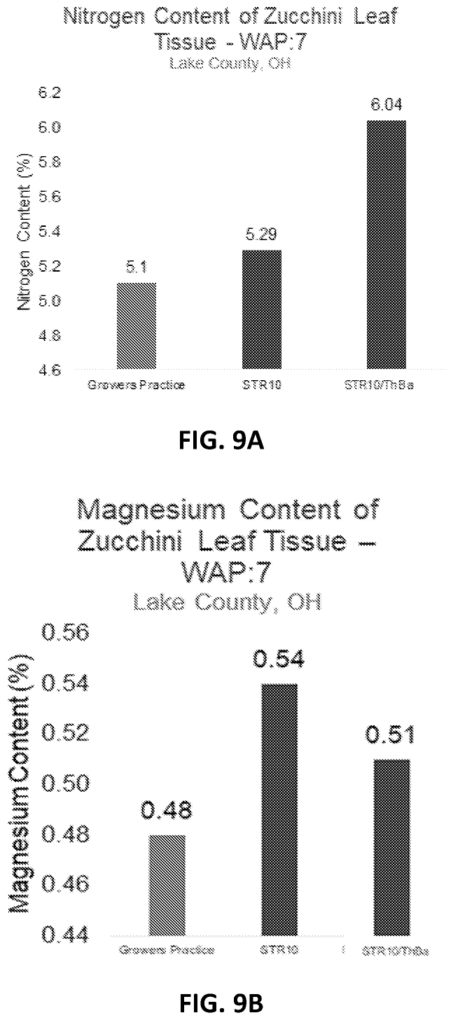

The nitrogen content of the leaves of each of the three groups was measured to determine general increase in nitrogen uptake. As depicted in FIG. 9A, the nitrogen content of the STR10 group was 0.19% greater than the control groups, and the nitrogen content of the STR10/ThBa group was 0.94% greater than the control group.

The magnesium content of the leaves of each of the three groups was also measured to determine general increase in magnesium uptake. As depicted in FIG. 9B, the magnesium content of the STR10 group was 0.06% greater than the control groups, and the magnesium content of the STR10/ThBa group was 0.03% greater than the control group.

1. A soil treatment composition for enhancing plant immune health, growth and/or yields, the composition comprising a Wickerhamomyces anomalus yeast and/or a growth by-product thereof.

2-4. (canceled)

5. The composition of claim 1, further comprising one or more additional microorganisms selected from Trichoderma spp., Bacillus amyloliquefaciens, Azotobacter vinelandii and Frateuria aurantia.

6. The composition of claim 1, further comprising one or more of a nano-fertilizer, kelp extract, fulvic acid, fumaric acid, chitin, a chitin derivative, humate and humic acid.

7. The composition of claim 1, formulated as a dry powder or dry granules.

8. The composition of claim 1, comprising 8-12% by volume Wickerhamomyces anomalus cells and 88-92% by volume fermentation substrate in which the Wickerhamomyces anomalus was produced.

9. The composition of claim 1, comprising at least 1×10⁶ CFU/ml of the Wickerhamomyces anomalus.

10. A method of enhancing plant immunity, health, growth and/or yields, the method comprising:
applying a Wickerhamomyces anomalus yeast and/or a growth by-product thereof and, optionally,
applying one or more of a nano-fertilizer, kelp extract, fulvic acid, fumaric acid, chitin, a chitin derivative, humate and humic acid,

11-12. (canceled)

13. The method of claim 10, wherein the yeast and/or growth by-product thereof is contacted directly with the plant's roots.

14. The method of claim 10, wherein the yeast and/or growth by-product thereof is contacted with soil in which the plant grows.

15. (canceled)

16. The method of claim 10, wherein the yeast and/or growth by-product thereof is applied to the plant and/or its surrounding environment using an irrigation system.

17. The method of claim 10, further comprising applying one or more additional microorganisms with the yeast and/or growth by-products thereof.

18. The method of claim 17, wherein the one or more additional microorganisms are selected from Trichoderma spp., Bacillus amyloliquefaciens, Azotobacter vinelandii and Frateuria aurantia.

19. The method of claim 10, used to enhance the health and/or growth of the plant's roots.

20. The method of claim 10, used to improve the immune health, vitality and productivity of the plant.

21. (canceled)

22. The method of claim 10, wherein the plant to which the soil treatment composition is applied has compromised immune health due to an infection from a pathogenic or biotic agent, or from an abiotic environmental stressor.

23. The method of claim 22, wherein the plant is a citrus plant affected by citrus greening disease and/or citrus canker disease.

24. The method of claim 10, used to improve one or more qualities of soil, wherein the yeast and/or growth by-product thereof are applied to soil.

25. The method of claim 24, used to improve water absorption and retention in dry soil.

26. The method of claim 24, used to improve water drainage and/or dispersal in waterlogged soil.

27. The method of claim 24, used to improve nutrient retention in soil.

28. The method of claim 10, used to enhance nutrient absorption in plant roots.

29. The method of claim 10, wherein the plant is a crop selected from citrus, tomato, sugar beet, soybean, zucchini, peanut, sod, corn, tobacco, potato, melon, sugarcane, grapes, lettuce, almond, onion, carrot, berries and cotton.

30. The method of claim 10, wherein the plant is a sod grass, turf grass, pasture grass, or tree.

31. The method of claim 10, wherein the plant is an ornamental plant selected from flowering plants, shrubs and bushes.

32. The method of claim 10, used for forestry and/or reforestation.

33-35. (canceled)
